(19) United States
(12) Patent Application Publication
(10) Pub. No.: US 2023/0293639 A1
(43) Pub. Date: Sep. 21, 2023

(54) SYSTEMS, DEVICES, COMPOSITIONS, AND METHODS FOR TREATING DIABETES

(71) Applicant: Abvance Therapeutics, Inc., Carlsbad, CA (US)

(72) Inventors: David Maggs, Chapel Hill, NC (US); Alan Cherrington, Nashville, TN (US); Soumitra S. Ghosh, San Diego, CA (US); Christopher A. Rhodes, San Diego, CA (US); Jui-Chen Lin, San Diego, CA (US); R. Maxwell Flaherty, Topsfield, MA (US); J. Christopher Flaherty, Nottingham, NH (US)

(21) Appl. No.: 17/928,740

(22) PCT Filed: Jun. 11, 2021
(86) PCT No.: PCT/US21/36937

§ 371 Date: Nov. 30, 2022

Related U.S. Application Data
(60) Provisional application No. 63/037,637, filed on Jun. 11, 2020.

(51) Int. Cl. A61K 38/28; A61K 38/26; A61P 3/10; A61M 5/142

(52) U.S. Cl. CPC A61K 38/28; A61K 38/26; A61P 3/10; A61M 5/142

(57) ABSTRACT

Systems, devices, compositions, and methods for treating diabetes are provided, including co-administering insulin and glucagon to a patient. Data collected from studies conducted to observe co-administration of insulin and glucagon to a patient are also provided. Optimal ratios of insulin and glucagon administration, as well as target doses for each, are also provided.

### RELATED APPLICATIONS

The present application claims priority to U.S. Provisional Patent Application Ser. No. 63/037,637, entitled “Systems, Devices, Compositions, and Methods for Treating Diabetes”, filed Jun. 11, 2020, the content of which is incorporated herein by reference in its entirety for all purposes.

The present application, while not claiming priority to, may be related to U.S. Provisional Patent Application Ser. No. 62/626,415, entitled “Systems, Devices, Compositions, and Methods for Treating Diabetes”, filed Feb. 5, 2018, the content of which is incorporated herein by reference in its entirety for all purposes.

### TECHNICAL FIELD

The present invention relates generally to systems, devices, compositions, and methods for treatment of a diabetic patient, and in particular to treatments including the delivery of multiple agents.

### BACKGROUND

Treatment of a diabetic patient often includes delivery of insulin, such as via injections via syringe or an insulin delivery pump. Hypoglycemia is the complication feared most by patients with insulin-treated diabetes, for both Type 1 and Type 2 diabetes, as well as for other more rare forms of diabetes (e.g. pancreatic diabetes). It is a major barrier to effective treatment because people under-dose insulin so as to avoid hypoglycemia. As a result, current treatments often result in inadequate glycemic control, involving undesired hypoglycemic and/or hyperglycemic events.

There is a need for systems, compositions, and methods that treat diabetic patients while reducing abnormal oscillations in blood glucose and hypoglycemic episodes.

### SUMMARY

Embodiments of the systems, devices, compositions, and methods described herein can be directed to systems, devices, compositions, and methods for treatment of a diabetic patient.

In some aspects, the present disclosure provides methods of treatment comprising co-administering insulin and glucagon to a patient, wherein the insulin and glucagon are co-administered, either separately or as a co-formulation, at an insulin:glucagon molar ratio between about 1:1 and about 8:1, and wherein the insulin and glucagon are administered in an amount therapeutically effective to simultaneously treat or inhibit hyperglycemia and to inhibit hypoglycemia. In some embodiments, the insulin and glucagon are co-administered at an insulin:glucagon molar ratio of between 1:1 and 6:1. In some embodiments, the insulin and glucagon are co-administered at an insulin:glucagon molar ratio of between 3:1 and 4:1.

According to an aspect of the preset inventive concepts, a method of treating diabetes in a patient comprising: co-administering insulin and glucagon to the patient, and the insulin and glucagon are co-administrated at an insulin:glucagon molar ratio of between 1:1 and 8:1, such as a ratio between 3:1 and 4:1. The insulin and glucagon can be administered in an amount therapeutically effective to simultaneously treat and/or inhibit at least one of hyperglycemia and hypoglycemia.

In some embodiments, the patient is hyperglycemic prior to co-administering the insulin and glucagon.

In some embodiments, co-administering the insulin and glucagon comprises administering to the patient a composition comprising insulin and glucagon. The composition can be stable at a temperature between 2° C. and 8° C. The composition can comprise a co-formulation comprising insulin and glucagon. The co-formulation can comprise insulin at a concentration between 1 mg/mL and 10 mg/mL, and glucagon at a concentration between 0.1 mg/mL and 1 mg/mL. The co-formulation can comprise insulin at a concentration between 3 mg/mL and 5 mg/mL, and glucagon at a concentration between 0.1 mg/mL and 0.8 mg/mL. The co-formulation can further comprise an aqueous buffer comprising glutamic acid. The aqueous buffer can further comprise one or more excipients selected from the group consisting of: CHAPS (3-[(3-Cholamidopropyl)dimethylammonio]-1-propanesulfonate hydrate); dextrose; and Tweens. The co-formulation can comprise a pH between 3.5 and 5.1. The co-formulation can further comprise an aqueous buffer comprising glycine. The aqueous buffer can further comprise an excipient, the excipient comprising CHAPS. The co-formulation can comprise a pH between 3.5 and 7.8. The co-formulation can further comprise an aqueous buffer comprising an organic co-solvent. The organic co-solvent can comprise NMP (N-methyl-2-pyrrolidone) or DMSO (dimethyl sulfoxide). The aqueous buffer can further comprise an excipient, the excipient comprising CHAPS. The co-formulation can further comprise one or more aqueous solvents. The one or more aqueous solvents can comprise one of the following buffer solutions: acetate; citrate; phosphate; or Tris. The one or more aqueous solvents can further comprise one or more excipients selected from the group consisting of: an amino acid, such as arginine, aspartic acid, cysteine, glutamic acid, glutamine, glycine, histidine, leucine, and lysine; a surfactant, such as Tweens, CHAPS, and Poloxamer 188; a cyclodextrin, such as HP-β-CD (2-Hydroxypropyl-beta-cyclodextrin) or M-β-CD (methyl-β-cyclodextrin); and/or a hydrophobic excipient, such as vitamin E TPGS (D-α-tocopheryl polyethylene glycol succinate) or nicotinamide.

In some embodiments, the co-administering the insulin and glucagon comprises the administering the insulin and glucagon subcutaneously. The insulin can comprise an insulin analog. The glucagon can comprise a glucagon analog. The patient can present with type-1, type-2, gestational, or other forms of diabetes mellitus. The patient can present with hypoglycemia associated autonomic failure.

According to another aspect of the present inventive concepts, a composition for treating diabetes in a patient comprises: a co-formulation of insulin and glucagon. The insulin can comprise a concentration between 1 mg/mL and 10 mg/mL and the glucagon can comprise a concentration between 0.1 mg/mL and 1 mg/mL. The molar ratio of insulin:glucagon can be between 1:1 and 8:1, such as a ratio between 3:1 and 4:1.

In some embodiments, the co-formulation comprises insulin at a concentration between 3 mg/mL and 5 mg/mL, and glucagon at a concentration between 0.1 mg/mL and 0.8 mg/mL.

In some embodiments, the co-formulation further comprises an aqueous buffer comprising glutamic acid. The aqueous buffer can further comprise one or more excipients selected from the group consisting of: CHAPS; dextrose; and Tweens. The co-formulation can comprise a pH between 3.5 and 5.1.

In some embodiments, the co-formulation further comprises an aqueous buffer comprising glycine. The aqueous buffer can further comprise an excipient, the excipient comprising CHAPS. The co-formulation can comprise a pH between 3.5 and 7.8.

In some embodiments, the co-formulation further comprises an aqueous buffer comprising an organic co-solvent. The organic co-solvent can comprise NMP, DMSO, propylene glycol, and/or glycerin. The aqueous buffer can further comprise an excipient, the excipient comprising CHAPS.

In some embodiments, the co-formulation further comprises one or more aqueous solvents. The one or more aqueous solvents can comprise one of the following buffer solutions: acetate; citrate; phosphate; or Tris. The one or more aqueous solvents can further comprise one or more excipients selected from the group consisting of: an amino acid, such as arginine, aspartic acid, cysteine, glutamic acid, glutamine, glycine, histidine, leucine, and lysine; a surfactant, such as Tweens, CHAPS, and Poloxamer 188; a cyclodextrin, such as HP-β-CD and M-β-CD; and/or a hydrophobic excipient, such as vitamin E TPGS and nicotinamide.

According to another aspect of the present inventive concepts, a system for a patient comprises: an infusion pump and a composition. The infusion pump can deliver the composition to the patient.

In some embodiments, the composition comprises a co-formulation of insulin and glucagon, and the pump comprises a single reservoir configured to store the co-formulation.

In some embodiments, the composition comprises insulin and glucagon, and the pump comprises a first reservoir for storing the insulin and a second reservoir for storing the glucagon. The pump can be configured to deliver the insulin and glucagon simultaneously. The pump can be configured to deliver the insulin and glucagon sequentially.

In some embodiments, the infusion pump comprises a functional element comprising one or more sensors. The one or more sensors can comprise a sensor selected from the group consisting of: a temperature sensor; a pressure sensor; an optical sensor; a strain gauge; an accelerometer; magnetic sensor; galvanic sensor; an ultrasonic sensor; a motion sensor; a flow sensor; physiologic sensor; blood gas sensor; glucose sensor; and combinations of one, two, three, or more of these. The infusion pump can comprise at least one reservoir, and the at least one sensor can be positioned proximate the at least one reservoir. The at least one sensor can be configured to detect undesired material within the at least one reservoir. The at least one sensor can be configured to monitor the temperature of the composition, and the system can be configured to alert a user if a temperature threshold has been exceeded. The at least one sensor can be configured to monitor the flow rate of the composition as the infusion pump delivers the composition to the patient.

In some embodiments, the infusion pump comprises a functional element comprising one or more transducers. The one or more transducers can comprise a transducer selected from the group consisting of: a heating element; a cooling element such as a Peltier cooling element; an agitator; a vibrational transducer; an audible transducer such as a speaker; a light producing element; and combinations of one, two, three, or more of these. The infusion pump can comprise at least one reservoir, and the at least one transducer can be positioned proximate the at least one reservoir. The at least one transducer can be configured to maintain the temperature of the composition within the at least one reservoir. The at least one transducer can be configured to agitate the composition within the at least one reservoir.

According to another aspect of the present inventive concepts, a method of treating diabetes in a patient comprises: administering insulin to the patient beginning at a first time period, and administering glucagon to the patient beginning at a second time period. The insulin and glucagon can be administrated at an insulin:glucagon molar ratio of between 1:1 and 8:1, such as a ratio between 3:1 and 4:1. The insulin and glucagon can be administered in an amount therapeutically effective to simultaneously treat and/or inhibit at least one of hyperglycemia and hypoglycemia.

In some embodiments, the second time period begins between 20 and 60 minutes after the first time period. The second time period can begin 30 minutes after the first time period.

In some embodiments, the insulin is administered at an average infusion rate of 7.29 pmol/kg/min and the glucagon is administered at an average infusion rate of 1.82 pmol/kg/min. The insulin and glucagon infusion rates can be decreased by half beginning at a third time period. The third time period can begin 180 minutes after the first time period.

According to another aspect of the present inventive concepts, a system for a patient comprises a syringe or an injectible pen device pre-filled with a composition. The syringe or injectible pen can deliver the composition to the patient.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a system for delivering a composition to a patient, comprising a single pumping device with a single reservoir, consistent with the present inventive concepts.

FIG. 1A illustrates a system for delivering a composition to a patient, comprising a single pumping device with two reservoirs, consistent with the present inventive concepts.

FIG. 1B illustrates a system for delivering a composition to a patient, comprising two pumping devices, consistent with the present inventive concepts.

FIGS. 2-11 illustrate data from studies conducted by applicant to support the development of an aqueous co-formulation of insulin and glucagon for use in infusion pumps that is stable at 2-8° C., consistent with the present inventive concepts.

FIG. 12 illustrates a study protocol conducted by the applicant in patients with Type 1 diabetes, consistent with the present inventive concepts.

FIGS. 13-20 illustrate data collected from studies conducted by the applicat in patients with Type 1 diabetes, consistent with present inventive concepts.

FIG. 21 illustrates a graph demonstrating optimized insulin to glucagon molar ratios and dosages, consistent with the present inventive concepts.

FIG. 22 illustrates a graph demonstrating the impact of various insulin to glucagon molar ratios on post-prandial glucose, consistent with the present inventive concepts.

FIG. 23 illustrates a graph demonstrating the impact of varous insulin and glucose absolute concentrations on post-prandial glucose, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

In this specification, unless explicitly stated otherwise, “and” can mean “or,” and “or” can mean “and.” For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold. within a threshold range of values and/or outside a threshold range of values, to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function. In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a patient anatomical parameter; and combinations of one or more of these. A functional element can comprise a fluid, such as an ablative fluid (as described hereabove) comprising a liquid or gas configured to ablate or otherwise treat tissue. A functional element can comprise a reservoir, such as an expandable balloon configured to receive an ablative fluid. A “functional assembly” can comprise an assembly constructed and arranged to perform a function, such as is described hereabove. In some embodiments, a functional assembly is configured to deliver energy and/or otherwise treat tissue (e.g. a functional assembly configured as a treatment assembly). Alternatively or additionally, a functional assembly can be configured to record one or more parameters, such as a patient physiologic parameter; a patient anatomical parameter; a patient environment parameter; and/or a system parameter. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, heat energy, cryogenic energy, chemical energy; mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

The term “insulin” where used herein is to be taken to include the hormone insulin and/or one or more insulin analogues (e.g. one or more insulin analogues known to one of skill in the art), such as, but not limited to, NPH insulin, insulin aspart, insulin glulisine, insulin lispro, insulin glargine, insulin determir, insulin degludec, hypurin bovine lente, hypurin bovine PZI, and/or hepatopreferential insulin.

The term “glucagon” where used herein is to be taken to include the hormone glucagon and/or one or more glucagon analogues (e.g. one or more glucagon analogues known to one of skill in the art), such as, but not limited to, Dasiglucagon (also known as ZP-4207, Zealand Pharmaceuticals), [Asp28] glucagon, [Asp28, Glu29] glucagon, and/or glucagon-Cex.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems, compositions, and methods for treating a diabetic patient, such as by delivering both insulin and glucagon simultaneously, or at least realtively simultaneously (“simultaneously” or “at the same time” herein). For example, simultaneous delivery includes sequential delivery of a volume of a first agent (e.g. insulin) and a volume of a second agent (e.g. glucagon), delivered in either order, that occurs within a time period of between 0.1 seconds and 90 minutes, a time period of between 0.1 seconds and 60 minutes, or a time period of between 0.1 seconds and 30 minutes.

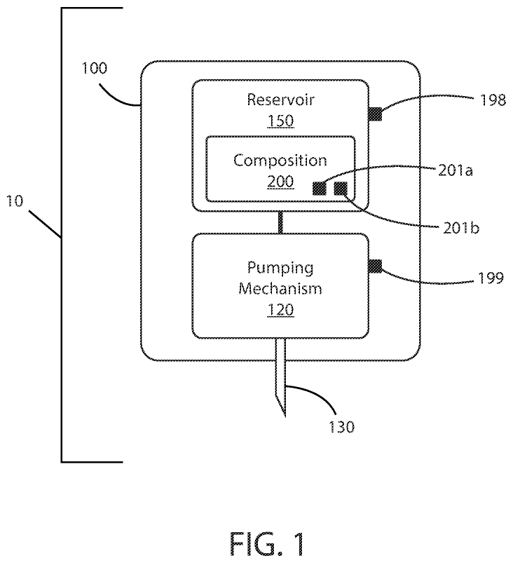

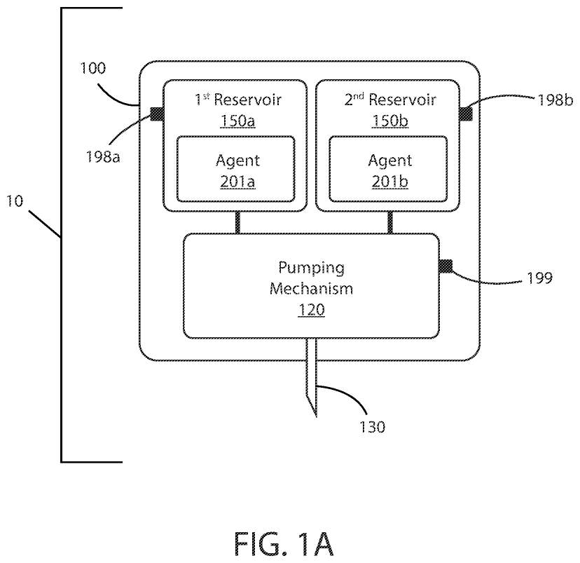

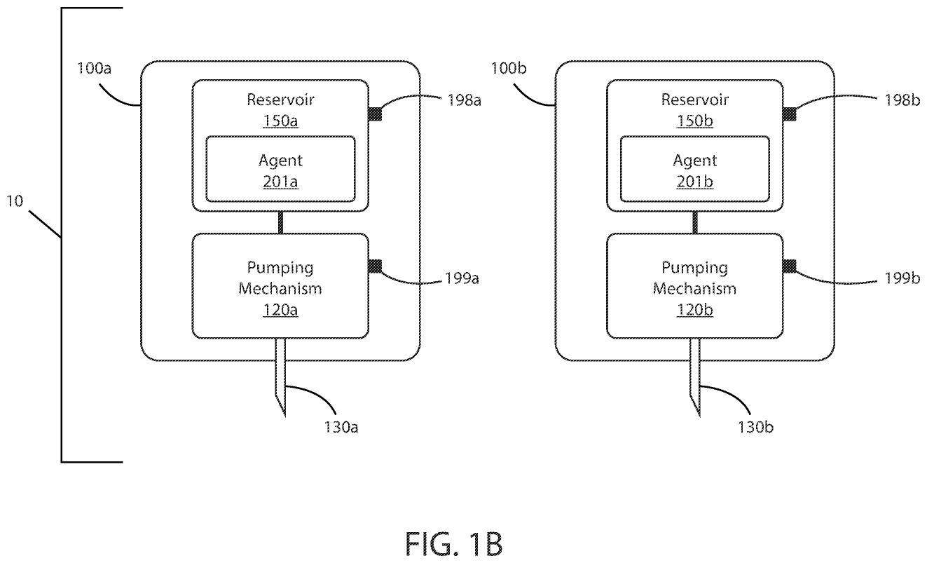

Referring now to FIG. 1, a system for treating a diabetic patient is illustrated, consistent with the present inventive concepts. System 10 comprises pump 100 and composition 200. Pump 100 can comprise reservoir 150, which can be used to surround, store, supply and/or otherwise provide (generally “provide” herein) composition 200, such as to allow prolonged and/or intermittent delivery of composition 200. Pump 100 can be configured to deliver composition 200 to one or more patient locations, such as when pump 100 delivers composition 200 into one or more of: subcutaneous tissue; a muscle; a vein; and/or an artery. Composition 200 can include two or more agents, such as when composition 200 comprises at least insulin (e.g. insulin and/or an insulin analogue, “insulin” herein) and glucagon and/or a glucagon analog (e.g. as a separate material or as a co-formulation or otherwise mixed state). Composition 200 can comprise a co-formulation or other mixture of at least two agents. Alternatively, composition 200 can comprise a first agent 201a (e.g. an agent including at least insulin and/or insulin analog) and a separate, second agent 201b (e.g. an agent including at least glucagon and/or a glucagon analog that is not mixed with the first agent). The ratio of the amount of first agent 201a and second agent 201b delivered to a patient by system 10 (e.g. an insulin/glucagon molar ratio, or I/G molar ratio) can be predetermined and/or otherwise controlled (e.g. controlled to a maximum, minimum, and/or within a range), such as to achieve a desired therapeutic benefit and/or lack of adverse events for the patient.

Pump 100 can comprise a pump positioned external to the patient, such as when pump 100 includes a fluid delivery element 130 comprising: an integrated needle (e.g. a needle positioned through the skin into a body location such as the subcutaneous (SQ) space, the intraperitoneal (IP) space, a vein, or an artery); an infusion set comprising a needle (e.g. a needle positioned through the skin into a body location such as the subcutaneous space, the intraperitoneal space, a vein, or an artery); and/or a catheter (e.g. a catheter positioned through the skin into a body location such as the subcutaneous space, the intraperitoneal space, a vein or an artery). Alternatively, pump 100 can comprise an implantable pump, such as when fluid delivery element 130 comprises a catheter, such as a catheter implanted in subcutaneous tissue. Pump 100 can comprise an implantable pump including a refill port accessible through the patient's skin via a needle.

Pump 100 can comprise one or more pumping mechanisms, such as a pumping mechanism 120 selected from the group consisting of: a syringe drive; a peristaltic pumping assembly; a rotary pump; a spring driven pump; and combinations of one or more of these. Reservoir 150 can comprise a single or multiple reservoirs, such as when reservoir 150 comprises one or more: syringes and/or chambers (e.g. compressible chambers).

In some embodiments, reservoir 150 comprises one or more syringes, such as a syringe that is pre-filled with composition 200.

In some embodiments, pump 100 comprises an injectible pen-construction comprising a reservoir 150 that is pre-filled with composition 200.

In some embodiments, composition 200 comprises a co-formulation of insulin and glucagon, such as when reservoir 150 comprises a single reservoir which provides the co-formulation. Composition 200 can comprise a co-formulation of glucagon and a hepato-preferential insulin. A hepato-preferential insulin has enhanced liver-focused action, where glucagon is an effective competitor under hypoglycemic conditions. Use of a hepato-preferential insulin in a co-formulated composition 200 can be configured to provide enhanced results as compared to a co-formulation including non-hepatopreferential insulin, as the impact of non-hepatopreferential insulin in a co-formulated form on muscle glucose uptake will become more prominent.

In some embodiments, composition 200 comprises insulin and glucagon with an I/G molar ratio below 12:1, such as an I/G molar ratio below about 8:1, below about 6:1, below about 4:1, or below about 3:1. In some embodiments, composition 200 comprises insulin and glucagon with an I/G molar ratio less than 10:1 but greater than 1:1. In some embodiments, composition 200 comprises insulin and glucagon with an I/G molar ratio between 3:1 and 4:1. The basal SQ insulin infusion rate varies between 0.2 and 0.6 mU/kg/min. Thus in order to have an I/G molar ratio of 3:1, system 10 would infuse glucagon SQ at a basal rate between 1.3 and 3.9 ng/kg/min. In some embodiments, system 10 infuses glucagon SQ at a basal rate between 1.0 and 4.0 ng/kg/min. Applicant's data demonstrate that an I/G molar ratio of approximately 3:1 provides hypoglycemic protection with little to no negative consequences regarding treatment of hyperglycemia when the insulin infusion rate is increased to cover a meal. The optimal I/G molar ratio can vary between 2:1 and 8:1, such as between 3:1 and 4:1. In some embodiments, anything less than 2:1 can result in glucagon levels that would increase postprandial hyperglycemia. In some embodiments, an I/G molar ratio greater than 8:1 or 10:1 can provide too little hypoglycemic protection. In addition, since glucagon and insulin analogs can have differing potencies from native human insulin and glucagon the optimal ratios may have to be further adjusted to take into account the differing potencies when insulin and glucagon analogs are used.

In some embodiments, pump 100 can comprise a pen-type injector, such as when pump 100 comprises a construction similar to insulin delivery pens that are commercially available. In these embodiments, activation of fluid delivery (e.g. by depressing a button or other control) causes both first agent 201a and second agent 201b to be delivered (e.g. a simultaneous and/or sequential delivery of agents 201a and 201b).

In some embodiments, pump 100 comprises a pump with dual reservoirs, such as is shown in FIG. 1A. Reservoir 150a can be configured to provide a first agent 201a (e.g. insulin) while reservoir 150b can be configured to provide a second agent 201b (e.g. glucagon). In these embodiments, composition 200 comprises agent 201a and separate (unmixed) agent 201b, collectively. Agents 201a and 201b can be mixed prior to entry into and delivery by a single pumping mechanism 120. The concentration of each of agents 201a and 201b stored in reservoirs 150a and 150b respectively, shall determine the ratio of the key components of each of the agents (e.g. to deliver a pre-determined molar ratio of insulin to glucagon as described herein). Alternatively, pump 100 can comprise two pumping mechanisms 120 (e.g. mechanisms 120a and 120b not shown but independently controllable mechanisms), such that the flow rate of pumping mechanism 120 can be configured (e.g. programmed or programmable) to achieve a desired ratio of delivery of agent 201a to agent 201b, independent of their relative concentrations.

In some embodiments, pump 100 comprises two pumps, pumps 100a and 100b, such as is shown in FIG. 1B. Pump 100a comprises reservoir 150a which can be configured to provide a first agent 201a (e.g. insulin). Pump 100b can comprise a second reservoir 150b which can be configured to provide a second agent 201b (e.g. glucagon). In these embodiments, composition 200 comprises agent 201a and separate (unmixed) agent 201b, collectively. The ratio of delivery of agent 201a to agent 201b is determined by both the concentration of each of the two agents 201a and 201b, as well as the programmed flow rates of each of the two pumps 100a and 100b (e.g. to deliver a pre-determined molar ratio of insulin vs glucagon as described herein).

In some embodiments, pump 100, pump 100a and/or pump 100b comprise one or more functional elements, such as functional elements 198 and 199 shown in FIG. 1, functional elements 198a, 198b, and 199 shown in FIG. 1A, and functional elements 198a, 198b, 199a, and 199b shown in FIG. 1B. Functional elements 198, 198a, and/or 198b (singly or collectively functional element 198) and/or functional elements 199, 199a, and/or 199b (singly or collectively functional element 199), can each comprise one, two, three, or more functional elements. In some embodiments, functional element 198 and/or 199 comprises one, two, three or more sensors, such as are described herebelow. Alternatively or additionally, functional element 198 and/or 199 can comprise one, two, three or more transducers, such as are also described herebelow.

Functional element 198 and/or 199 can comprise a sensor selected from the group consisting of: a temperature sensor; a pressure sensor; an optical sensor; a strain gauge; an accelerometer; magnetic sensor; galvanic sensor; an ultrasonic sensor; a motion sensor; a flow sensor; physiologic sensor; blood gas sensor; glucose sensor; and combinations of one, two, three, or more of these. In some embodiments, functional element 198 is positioned proximate reservoir 150 (as shown in FIGS. 1, 1A and 1B), and comprises one or more sensors (e.g. one or more optical sensors) configured to detect an undesired material (e.g. contaminant or precipitate) within a reservoir 150, such that system 10 can provide an alert to a user of system 10 if the undesired material is detected. In some embodiments, functional element 198 and/or 199 comprises one, two, or more temperature sensors configured to monitor the temperature of first agent 201a and/or second agent 201b, such that system 10 can provide an alert to a user of system 10 if the temperature exceeds (or has previously exceeded) a temperature threshold (e.g. an undesirably cold temperature such as a freezing temperature and/or an undesirably warm temperature). In some embodiments, functional element 198 and/or 199 comprises one, two, or more flow sensors configured to monitor the flow of first agent 201a and/or second agent 201b (e.g. monitor the flow exiting fluid delivery element 130), such that system 10 can provide an alert to a user of system 10 if the flow rate is at an undesired level (e.g. a rate above or below a desired rate). In these embodiments, system 10 can include an algorithm configured to monitor the flow rates of first agent 201a and second agent 201b and provide an alert based on an analysis of the flow rates (e.g. an analysis of both flow rates that is correlated to a desired range of acceptable ratios of first agent 201a and second agent 201b delivered to the patient).

Functional element 198 and/or 199 can comprise a transducer selected from the group consisting of: a heating element; a cooling element such as a peltier cooling element; an agitator; a vibrational transducer; an audible transducer such as a speaker (e.g. to provide an alert); a light producing element (e.g. a light emitting diode or other light producing element configured to provide information to a user and/or to deliver light to agents 201a and/or 201b); and combinations of one, two, three, or more of these. In some embodiments, functional element 198 is positioned proximate reservoir 150 (as shown in FIGS. 1, 1A and 1B), and comprises one or more tranducers (e.g. one or more peltier elements) configured to cool the contents of reservoir 150 (e.g. maintain agents 201a and/or 201b at a temperature that provides prolonged stability to the agent). In these embodiments, functional element 198 can be configured to maintain agents 201a and/or 201b at a temperature below body temperature and/or below room temperature. In some embodiments, functional element 198 is positioned proximate reservoir 150 (as shown in FIGS. 1, 1A and 1B), and comprises one or more tranducers configured to deliver heat to the contents of reservoir 150. In these embodiments, functional element 198 and/or 199 can (also) comprise a temperature sensor, such that system 10 can prevent agents 201a and/or 201b from freezing (e.g. heat is delivered when temperature proximate reservoir 150 approaches a freezing level). In some embodiments, functional element 198 is positioned proximate (e.g. within) reservoir 150 and comprises one or more tranducers configured to agitate or otherwise cause mixing of the contents of reservoir 150, such as to mix components of agent 201a and/or 201b, and/or to mix agents 201a and 201b.

In some embodiments, pump 100 and/or another component of system 10 comprises an array of at least one sensor and/or at least one transducer configured to maintain the integrity of agents 201a and/or 201b, and/or to ensure accuracy of delivery of these agents (e.g. to maintain temperature and/or ensure proper amounts are delivered to the patient).

Composition 200 can be configured such that its glucagon and insulin work in concert to closely regulate blood glucose. Insulin promotes the removal of glucose from blood to muscle and fat tissue and also inhibits the production of glucose by the liver, thereby lowering blood glucose levels. Glucagon stimulates hepatic glucose production, which is released into the bloodstream to elevate blood glucose. In people with diabetes, both the beta cell, which secretes insulin, and the alpha cell, which secretes glucagon, become defective. This issue manifests as insulin deficiency which leads to decreased glucose utilization and increased glucose production. At the same time, glucagon excess also results in increased glucose production. It is not surprising, therefore, that current therapeutic approaches have focused on enhancing insulin secretion and action, and blocking glucagon secretion and action.

One of the major barriers to the effective treatment of people with diabetes is their defective ability to respond to hypoglycemia with a normal glucagon response, particularly in people with Type 1 diabetes (T1D). This deficiency makes patients with T1D more susceptible to hypoglycemia than the normal (non-diabetic) individual. As a result of this defect, the blood glucose level in these patients oscillates wildly, exhibiting both marked hyperglycemia and hypoglycemia, thereby making it very difficult for such patients to adequately control their blood glucose level with insulin treatment alone. Recently, investigators, using closed loop insulin pumps, have come to the conclusion that using insulin at times of high blood glucose and glucagon at times of low blood glucose can reduce the magnitude of both hypoglycemia and hyperglycemia (e.g. as described in publications by Russel, et al in New England Journal of Medicine, June 2014 and Bakhtiani, et al, in Diabetes, Obesity and Metabolism, 2013). Notwithstanding the use of real-time glucose sensors and sophisticated algorithms to trigger either insulin or glucagon infusion in closed loop systems, there is still a significant unmet need to reduce such oscillations in blood glucose and to minimize hypoglycemic episodes.

Composition 200 comprises a particular relationship (e.g. ratio) between the quantities of insulin and glucagon to achieve a beneficial therapeutic effect while minimizing hypoglycemia. Composition 200 can comprise such a ratio and/or otherwise be configured to avoid complications from hyperinsulinemia and/or hyperglucagonemia. In some embodiments, system 10 and composition 200 are configured to provide sufficient glucagon to the patient to be able to protect against hypoglycemia risk in the context of hyperinsulinemia or increased insulin delivery. System 10 can provide glucagon to the patient at a rate of approximately 2 ng/kg/min. System 10 can provide glucagon at a rate of more than 0.5 ng/kg/min, or more than 0.75 ng/kg/min, such as to protect against hypoglycemia when insulin levels are elevated or infusion rate is increased. Alternatively or additionally, system 10 can provide a glucagon infusion rate less than 20 ng/kg/min, so as to avoid increasing the risk of metabolic derangement and/or cardiovascular toxicity from excess glucagon. In some embodiments, system 10 and composition 200 provide an infusion rate of insulin configured to maintain glucose homeostasis in the face of the previously defined levels of glucagon infusion. Composition 200 can comprise a ratio between 1:1 and 10:1 of human insulin:human glucagon, such as a ratio of between 1:1 and 8:1, such as between 1:1 and 6:1, such as a ratio of between 3:1 and 4:1 (e.g. to lower glucose effectiveness as well as reduce risk of hypoglycemia). Composition 200 can comprise a molar ratio of less than 12:1, such as to achieve sufficient hypoglycemic risk reduction at the glucagon infusion rate that can protect against hypoglycemia. In some embodiments, system 10 administers less than 3.2 mU/kg/min of insulin (e.g. human insulin), because excess insulin may not be overcome by any amount of glucagon at that administered infusion rate.

In some embodiments, system 10 provides glucagon at a minimum rate that is configured to be sufficient for protection from hypoglycemia under conditions of hyperinsulinemia without causing toxicity from hyperglucagonemia, such as a rate above 10 ng/kg/min or a rate above 20 ng/kg/min that could be delivered subcutaneously. In some embodiments, composition 200 comprises insulin:glucagon at a ratio configured to allow improved glucose homeostasis in the context of the glucagon delivered by system 10 via composition 200. By administering a composition 200 comprising a fixed molar ratio, system 10 provides protection from hypoglycemia even if insulin will be bolused by system 10 (e.g. via pump 100).

Applicant has conducted studies, which have shown that the way in which glucagon and insulin interact to control liver glucose production is influenced by the prevailing plasma glucose level. For instance, raising both the insulin and glucagon levels 4-fold on a molar basis under euglycemic conditions results in insulin action dominating glucagon action. In fact, the ability of a 4-fold rise in glucagon to stimulate hepatic glucose production is reduced by 80% when the insulin level also rises 4-fold. The reverse occurs under hypoglycemic conditions. Applicant has shown that under hypoglycemic conditions, glucagon becomes 3 times more effective in the presence of low glucose than under euglycemic conditions, even in the presence of high insulin levels. It has been demonstrated that hypoglycemia disengages insulin signaling in the liver thus allowing glucagon to work better. This improved glucagon effectiveness leads to the paradoxical possibility that co-administration of insulin and glucagon provides a therapeutic advantage.

Applicant acknowledges that while insulin and glucagon have opposing actions in regulating blood glucose, applicant's studies demonstrate that their interaction is glucose dependent. At high blood glucose concentrations, insulin dominates glucagon action, making the latter much less effective in stimulating liver glucose metabolism. At low blood glucose concentrations, glucagon dominates insulin action, making the latter much less effective in inhibiting hepatic glucose metabolism. Consequently, insulin and glucagon, when present in the circulation at certain absolute concentrations (e.g. concentrations above a minimum and/or below a maximum) and/or at certain insulin/glucagon molar ratios (e.g. ratios above a minimum and/or below a maximum), will have a different effect on the liver depending on the prevailing blood glucose level. As such, insulin and glucagon, when present at a certain molar ratio range, can be effective at limiting excessive blood glucose excursions (i.e. manifesting glucose responsiveness in the opposing actions of the two peptides). An optimal I/G molar ratio (e.g. an optimized I/G molar ratio) can lead to an increased time in range for blood glucose since postprandial glucose rise would be restrained and meal associated hypoglycemia would be prevented. Based on applicant's studies, an optimal I/G molar ratio lies between 1:1 and 8:1, such as between 3:1 and 4.5:1. As described herein, an I/G molar ratio of 4:1 comprised of delivering insulin at a rate between 3.65 pmol/kg/min to 10.95 pmol/kg/min and delivering glucagon at a rate between 0.91 pmol/kg/min to 2.74 pmol/kg/min can control post-prandial glycemia and limit post-meal hypoglycemia. The utility of this molar ratio relationship between the two hormones is limited by the absolute amounts of the hormones delivered to the patient. Since insulin stimulates muscle glucose uptake and glucagon does not, using too much insulin (e.g. greater than 3.0 mU/kg/min) may cause hypoglycemia that cannot be prevented by glucagon regardless of the I/G molar ratio. Likewise, using too much glucagon may also lead to difficulties. At high glucagon doses (e.g. delivered at a rate greater than 8 ng/kg/min), it can have an ionotropic effect on the heart, it can slow gastrointestinal motility and it can elicit detrimental effects in the central nervous system. An insulin:glucagon relationship, that offers a combinatorial glucose responsive pharmacological platform within certain dosing limits, offers improved clinical utility for subjects with diabetes already requiring insulin treatment. The described molar ratio relationship will manifest similarly with insulin and glucagon analog molecules that have a pharmacodynamic profile that approximates native insulin and glucagon.

The present inventive concepts described herein teach that co-administration or co-formulation of insulin and glucagon in the correct proportion allows more aggressive and yet safe treatment of T1D patients, providing enhanced long-term control of blood sugar levels. During eating and elevated blood glucose, the systems, compositions and methods of the present inventive concepts provide a prandial dose of insulin and glucagon in which the two are increased proportionately, where the impact of the extra insulin overrides the impact of the extra glucagon. During periods of low blood sugar, on the other hand, elevated insulin would be less effective at the liver, allowing the extra glucagon to drive increased glucose production thereby limiting hypoglycemia and reducing the need for sympathetic nervous system activation. The present inventive concepts provide co-administration and/or co-formulation of glucagon and insulin that limits these glycemic excursions, thereby improving HbAlc levels and reducing diabetic complications of the patient.

The present disclosure provides: using co-infusions of defined ratios of insulin and glucagon for control of blood glucose; using a co-formulated insulin glucagon mixture for control of blood glucose; use of an insulin and glucagon mixture to reduce hypoglycemia associated autonomic failure (HAAF); use of an insulin glucagon mixture to prevent insulin-mediated weight gain; use of an insulin glucagon mixture to limit fat accumulation in the liver. The present inventive concepts safely reduce glycemic variability in T1D patients, thereby allowing more aggressive treatment which leads to improved HbAlc levels and reduced complications to the patient. As described herein, insulin of the present inventive concepts can include insulin, analogues of insulin, and a preferentially biased insulin, such as hepato-preferential insulin.

Normally, insulin and glucagon are secreted into the hepatic portal vein such that the liver is exposed to a level 2 to 3-fold greater than any other tissues. After an overnight fast, the basal I/G molar secretion ratio is approximately 10:1, but it can vary from a low level (e.g. approximately 0) to a high level (e.g. approximately 240) in a state of hypoglycemia or hyperglycemia respectively. In applicant's preliminary experiments, it was shown that when the two hormones are infused peripherally, a molar ratio of approximately 3:1 to 4:1 can be used to maintain normal fasting glucose metabolism when the glucagon infusion rate was 1.6 ng/kg/min. Studies included examining the ability of a rise in insulin to cause hypoglycemia or prevent hyperglycemia, by delivering composition 200 with an I/G ratio of approximately 20:1 and approximately 4:1.

In some embodiments, composition 200 comprises a particle suspension of insulin in aqueous buffer and glucagon in solution, or a particle suspension of glucagon in aqueous buffer and insulin in solution. For example, composition 200 can comprise insulin in solution of 10 mm phosphate buffer with a pH of 7.4 and a nano- or micro-particle suspension of glucagon. In some embodiments, composition 200 comprises a particle suspension of both insulin and glucagon in an aqueous buffer. For example, composition 200 can comprise insulin and glucagon particle suspensions in one of the following aqueous buffers: 50 mM acetate buffer with a pH of 4.0; 50 mM citrate buffer with a pH between 4 and 5; or 50 mm phosphate buffer with a pH of 6. In some embodiments, composition 200 comprises a particulate composition, such as: nano- and micro-particles made from pure drug; a drug salt; lipid nanoparticles; and/or other compositions that render the suspended drug insoluble in the vehicle, and known to those practiced in the art of particle formation.

In some embodiments, composition 200 comprises a particle suspension of insulin in non-aqueous system and glucagon in solution, or a particle suspension of glucagon in non-aqueous system and insulin in solution. For example, composition 200 can comprise glucagon in solution of glycerol and a particle suspension of insulin. In some embodiments, composition 200 comprises a particle suspension of both insulin and glucagon in non-aqueous system. For example, composition 200 can comprise insulin and glucagon particle suspensions in polyethylene glycol (PEG), benzyl benzoate, or ethanol. In some embodiments, composition 200 comprises a particle suspension of both insulin and glucagon in oils or medium chain triglycerides. For example, composition 200 can comprise insulin and glucagon particle suspensions in corn oil, cottonseed oil, peanut oil, sesame oil, soybean oil, ethyl oleate, ethyl palmitate, glycerol oleate, isopropyl myristate, isopropyl palmitate, miglyol or neobee.

In some embodiments, composition 200 comprises an aqueous co-formulation of glucagon and insulin (e.g. at a fixed molar ratio) that also includes a molecular biochaperone. The biochaperone can be dextran-based molecules functionalized with natural amino acid and carboxylate, and it can mimic the properties of heparin for protein stabilization and solubility. The biochaperone forms molecular complexes with each hormone via electrostatic and hydrophilic interactions, and enables stable coformulation of insulin and glucagon at neutral pH.

In some embodiments, composition 200 comprises an aqueous co-formulation of insulin and glucagon (e.g. at a fixed molar ratio) and includes one or more sugars, such as dextrose, such as at concentrations ranging from 5-20%.

In some embodiments, composition 200 comprises an aqueous co-formulation of insulin and glucagon (e.g. at a fixed ratio) that also includes a surfactant excipient. The surfactant can comprise 1-Myristoyl-2-hydroxy-sn-glycero-3-phosphocholine or N-dodecyl-β-D-maltoside.

In some embodiments, composition 200 comprises an aqueous co-formulation of insulin and glucagon (e.g. at a fixed molar ratio) that are stabilized in solution with the addition of cucurbit[7]uril (CB[7])—PEG conjugate excipient via noncovalent, supramolecular host—guest interactions.

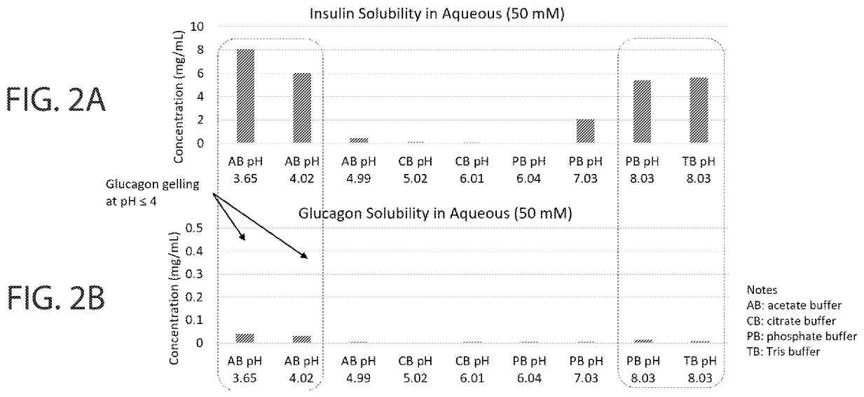

Referring now to FIGS. 2-7, results from studies conducted by applicant are presented, consistent with the present inventive concepts. These studies were conducted to support the development of a co-formulation (e.g. composition 200) of insulin and glucagon containing aqueous solution for use in infusion pumps, such as pump 100 of system 10 described hereabove, that is stable at 2-8° C. Challenges included glucagon's decreased solubility at all pH levels, as well as glucagon's gelling and aggregation at a pH of between 3 and 5. Aggregated glucagon is cytotoxic at high concentration and its action in vivo is delayed compared with fresh glucagon. An additional challenge included insulin's greater solubility at both low and high pH levels. With an objective of preventing aggregation and gelling, as well as improving solubility and stability, several approaches were employed in the development of the aqueous solution: modification of the buffer solution (e.g. acetate, citrate, phosphate, Tris) and the pH level (e.g. between 3.5 and 8.0); addition of an amino acid (e.g. arginine, aspartic acid, cysteine, glutamic acid, glutamine, glycine, histidine, leucine, lysine); addition of a surfactant to prevent aggregation and fibrillation (e.g. Tweens, 3-[(3-Cholamidopropyl)dimethylammonio]-1-propanesulfonate (CHAPS), Poloxamer 188); addition of a cyclodextrin for complexation (e.g. hydroxypropyl-β-cyclodextrin (HP-β-CD), methyl-β-cyclodextrin (M-β-CD)); and/or addition of hydrophobic excipients (e.g. d-α-tocopherol polyethylene glycol succinate (vitamin E TPGS), nicotinamide).

These studies were conducted with a target concentration of insulin of between 1 to 10 mg/mL and a target concentration of glucagon of between 0.1 and 1.0 mg/mL, more specifically an insulin concentration from 3 to 5 mg/mL and a glucagon concentration from 0.1 to 0.8 mg/mL. A basal insulin rate of U-100 between 10-20 μL/hr, and an I/G molar ratio of 3:1 to 16:1 with a fixed insulin dose were assumed. As described here below, insulin and glucagon achieve target solubility (e.g. greater than 3.5 mg/mL for insulin, and greater than 0.5 mg/mL for glucagon) in aqueous buffer systems comprising less than 30% co-solvents. The addition of co-solvents, including propylene glycol (PG), glycerol, and surfactants (e.g. CHAPS, vitamin E TPGS) improve solubility, as well.

FIGS. 2A and B illustrate data related to the solubility of insulin and glucagon, respectively, in various aqueous buffer solutions. The aqueous buffer solutions comprise acetate buffer (AB), citrate buffer (CB), phosphate buffer (PB), or Tris buffer (TB). The aqueous buffer solutions further comprise a pH level of between 3.65 and 8.03. The concentrations of insulin and glucagon in the supernatant were analyzed by RP-HPLC. Insulin and glucagon demonstrated improved solubility at a pH of less than 4 and greater than 7. As shown in FIG. 2A, insulin demonstrated an improved solubility of greater than 5 mg/mL in aqueous buffer solutions comprising: AB with a pH of 3.65; AB with a pH of 4.02; PB with a pH of 8.03; and TB with a pH of 8.03. Insulin demonstrated a poor solubility of less than 3 mg/mL in aqueous buffer solutions comprising: AB with a pH of 4.99; CB with a pH of 5.02; CB with a pH of 6.01; PB with a pH of 6.04; and PB with a pH of 7.03. As shown in FIG. 2B, glucagon demonstrated a poor solubility of less than 0.1 mg/mL in each of the aqueous buffer solutions. Additionally, glucagon gelling was observed in aqueous buffer solutions comprising AB with a pH of 3.65 and 4.02.

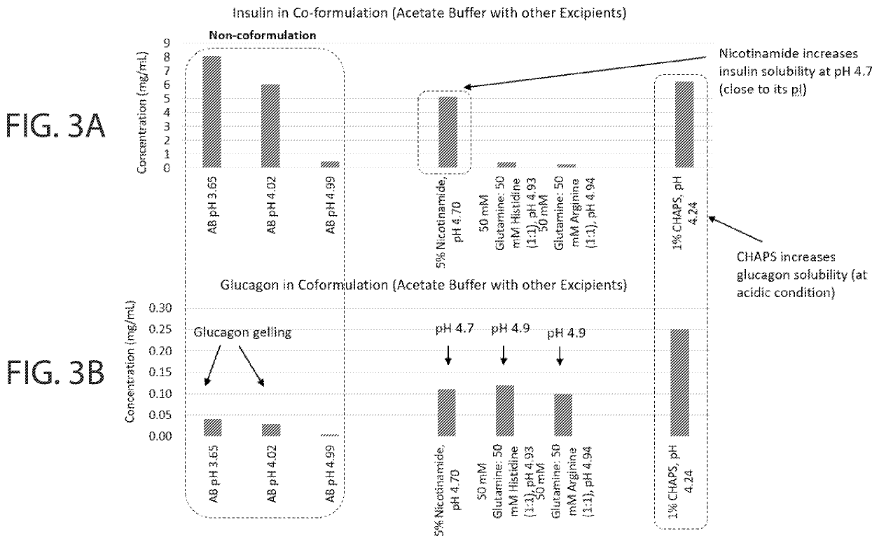

FIGS. 3A and B illustrate data related to the solubility of insulin and glucagon, respectively, in various co-formulations comprising an aqueous buffer solution with the addition of an excipient. The aqueous buffer solution comprises AB with a pH of between 4 and 5, and further comprises at least one of the following excipients: nicotinamide; one or more amino acids; and CHAPS. The concentrations of insulin and glucagon in the supernatant were analyzed by RP-HPLC. As shown in FIG. 3A, insulin demonstrated an improved solubility of greater than 5 mg/mL in non-coformulations (e.g. without the addition of an excipient) comprising AB with a pH of 3.65 and 4.02. Insulin demonstrated a poor solubility of less than 1 mg/mL in AB with a pH of 4.99. Insulin demonstrated an improved solubility of greater than 4 mg/mL in an aqueous buffer solution comprising AB with 5% nicotinamide and a pH of 4.70 (e.g. close to insulin pI of 5.4). Insulin also demonstrated an improved solubility of greater than 6 mg/mL in an aqueous buffer solution comprising AB with 1% CHAPS and a pH of 4.24. Insulin demonstrated a poor solubility of less than 1 mg/mL in an aqueous buffer solution comprising: AB with 50 mM of glutamine, 50 mM of histidine, and a pH of 4.93; and AB with 50 mM of glutamine, 50 mM of arginine, and a pH of 4.94.

As shown in FIG. 3B, glucagon demonstrated very poor solubility of less than 0.05 mg/mL in non-co-formulations (e.g. without the addition of an excipient) comprising AB with a pH of 3.65, 4.02, and 4.99. Additionally, glucagon gelling was observed in the non-co-formulations comprising AB with a pH of 3.65 and 4.02. Glucagon demonstrated improved solubility of equal or greater than 0.10 mg/mL in an aqueous buffer solution comprising: AB with 5% nicotinamide and a pH of 4.70; AB with 50 mM glutamine and 50 mM histidine, and a pH of 4.93; AB with 50 mM glutamine and 50 mM arginine, and a pH of 4.94. Glucagon demonstrated an unexpected improved solubility of greater than 0.2 mg/mL in an aqueous buffer solution comprising AB with 1% CHAPS and a pH of 4.24 (e.g. at acidic condition). Further, insulin displays improved solubility (−6 mg/ml) under the same acidic conditions with 1% CHAPS as excipient, as shown in FIG. 3A. This is in contrast to the poor solubility of glucagon in acidic buffers without the addition of any excipient, as shown in FIG. 2A.

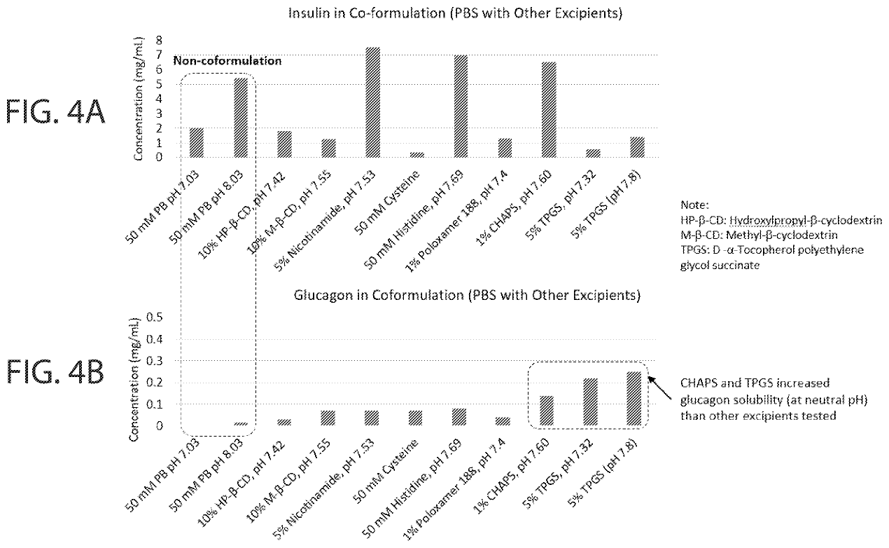

FIGS. 4A and B illustrate data related to the solubility of insulin and glucagon, respectively, in various co-formulations comprising an aqueous buffer solution with the addition of an excipient. The aqueous buffer solution comprises phosphate buffer saline (PBS) with a pH range between 7.03 and 8.03, and further comprises at least one of the following excipients: H-β-CD; M-β-CD; nicotinamide; one or more amino acids; poloxamer 188; CHAPS; and vitamin E TPGS. The concentrations of insulin and glucagon in the supernatant were analyzed by RP-HPLC. As shown in FIG. 4A, insulin demonstrated an improved solubility of greater than 5 mg/mL in a solution comprising 50 mM PBS with a pH of 8.03 (e.g. without the addition of an excipient), and demonstrated a poor solubility of less than 2.5 mg/mL in a buffer comprising 50 mM PBS with a pH of 7.03. Insulin demonstrated improved solubility of greater than 6 mg/mL in aqueous buffer solutions comprising: PBS with 5% nicotinamide with a pH of 7.53; PBS with 50 mM histidine and a pH of 7.69; and PBS with 1% CHAPS and a pH of 7.60. Insulin demonstrated a poor solubility of less than 2 mg/mL in aqueous buffer solutions comprising: PBS with 10% HP-β-CD and a pH of 7.42; PBS with 10% M-β-CD and a pH of 7.55; PBS with 50 mM cysteine and a pH of 7.4; PBS with 1% poloxamer 188 and a pH of 7.7; PBS with 5% vitamin E TPGS and a pH of 7.32; and PBS with 5% vitamin E TPGS and a pH of 7.8.

As shown in FIG. 4B, glucagon demonstrated a poor solubility of less than 0.05 mg/mL in buffers without the addition of an excipient, such as those comprising 50 mM PBS with a pH of 7.03 and 8.03. The solubility of glucagon is improved to greater than 0.2 mg/mL in an aqueous buffer solution comprising PBS with 5% vitamin E TPGS and a pH of 7.32, and an aqueous buffer solution comprising PBS with 5% vitamin E TPGS and a pH of 7.8. Further, glucagon demonstrated an improved solubility of greater than 0.1 mg/mL in an aqueous buffer solution comprising PBS with 1% CHAPS and a pH of 7.60. Glucagon demonstrated a poor solubility of less than 0.1 mg/mL in an aqueous buffer solution comprising: PBS with 10% HB-β-CD and a pH of 7.42; PBS with 10% M-β-CD and a pH of 7.55; PBS with 5% nicotinamide with a pH of 7.53; PBS with 50 mM cysteine and a pH of 7.4; PBS with 50 mM histidine and a pH of 7.69; and PBS with 1% poloxamer 188 and a pH of 7.4.

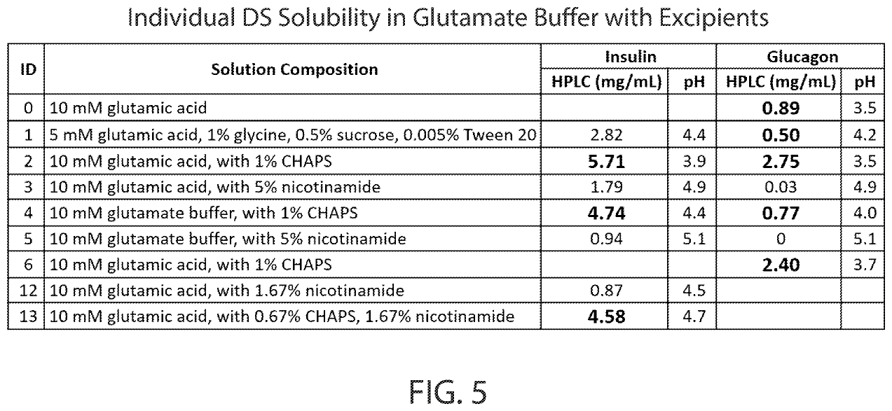

FIG. 5 illustrates data related to the solubility of insulin (e.g. up to approximately 5 mg/mL) and glucagon (e.g. up to approximately 2.5 mg/mL), respectively, in various acidic glutamate buffer solutions with the addition of an excipient. The glutamate buffer (GB) solution comprises a pH of between 3.5 and 5.1, and further comprises at least one of the following excipients: nicotinamide; one or more amino acids; sucrose; Tween 20; and CHAPS. The concentrations of insulin and glucagon in the supernatant were analyzed by RP-HPLC. Insulin demonstrated an improved solubility of greater than 4 mg/mL in a glutamate solution comprising: 10 mM glutamic acid with 1% CHAPS and a pH of 3.9; 10 mM glutamate buffer with 1% CHAPS and pH of 4.4; and 10 mM glutamic acid with 0.67% CHAPS and 1.67% nicotinamide, and a pH of 4.7. Insulin demonstrated a poor solubility of less than 3 mg/mL in a glutamate solution comprising: 5 mM glutamic acid with 1% glycine, 0.5% sucrose, and 0.005% Tween 20, and a pH of 4.4; 10 mM glutamic acid with 5% nicotinamide and a pH of 4.9; 10 mM glutamate buffer with 5% nicotinamide and a pH of 5.1; and 10 mM glutamic acid with 1.67% nicotinamide and a pH of 4.5. CHAPS increases insulin solubility in glutamate buffer of pH 4.7 (e.g. close to insulin pI).

Glucagon demonstrated a significantly improved solubility of 0.89 mg/mL in a buffer without the addition of an excipient comprising 10 mM glutamic acid with a pH of 3.5. The solubility of glucagon was adversely affected, namely 0.03 mg/mL or less, in a glutamate solution comprising 10 mM glutamic acid with 5% nicotinamide and a pH of 4.9, and a glutamate solution comprising 10 mM glutamate buffer with 5% nicotinamide and a pH of 5.1. In contrast, glucagon demonstrated an improved solubility of 0.5 mg/mL or greater in a glutamate solution comprising: 5 mM glutamic acid with 1% glycine, 0.5% sucrose, and 0.005% Tween 20, and a pH of 4.2; 10 mM glutamic acid with 1% CHAPS and a pH of 3.5; and 10 mM glutamate buffer with 1% CHAPS and pH of 4.0. Notably, the solubility of glucagon reached 2.4 mg/mL and 2.75 mg/mL, respectively, withl0 mM glutamic acid with 1% CHAPS, and a pH of 3.7 and 3.5.

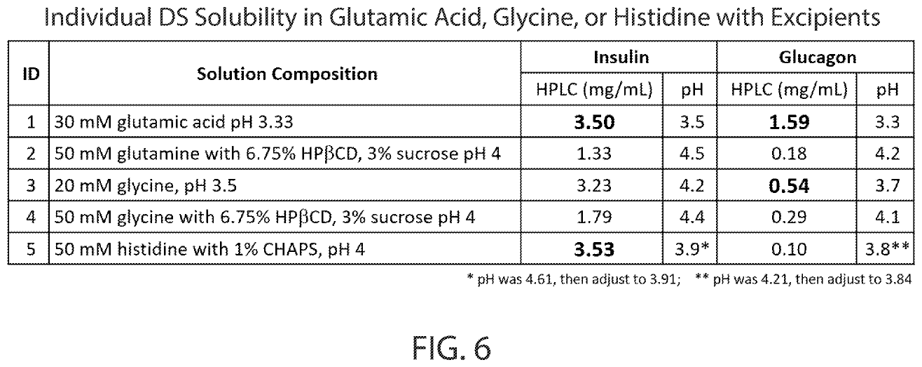

FIG. 6 illustrates data related to the solubility of insulin (e.g. up to approximately 3.5 mg/mL) and glucagon (e.g. up to approximately 1.6 mg/mL), respectively, in various aqueous solutions comprising glutamic acid, glycine, or histidine and the addition of an excipient. The glutamic acid solutions comprise a pH between 3.33 and 4.0, the glycine solutions comprise a pH between 3.5 and 4.0, and the histidine solutions comprise a pH of 4, and each solution further comprises at least one of the following excipients: HP-β-CD; sucrose; and CHAPS. The concentrations of insulin and glucagon in the supernatant were analyzed by RP-HPLC. Insulin demonstrated an improved solubility of 3.50 mg/mL in solution comprising 30 mM glutamic acid with a pH 3.3, and demonstrated an improved solubility of 3.53 mg/mL in solution comprising 50 mM histidine with 1% CHAPS with a pH of 4. Insulin demonstrated a poor solubility of 3.23 mg/mL or less in solutions comprising: 50 mM glutamine with 6.75% HP-β-CD and 3% sucrose, and with a pH of 4; 20 mM glycine with a pH of 3.5; and 50 mM glycine with 6.75% HP-β-CD and 3% sucrose, and with a pH of 4.

Glucagon demonstrated an improved solubility of 1.59 mg/mL in solution comprising 30 mM glutamic acid with a pH 3.3, and demonstrated an improved solubility of 0.54 mg/mL in solution comprising 20 mM glycine with a pH of 3.5. Glucagon demonstrated a poor solubility of 0.29 mg/mL or less in solutions comprising: 50 mM glutamine with 6.75% HP-β-CD and 3% sucrose, and with a pH of 4; 50 mM glycine with 6.75% HP-β-CD and 3% sucrose, and with a pH of 4; and 50 mM histidine with 1% CHAPS with a pH of 4. In regard to the solutions comprising 50 mM histidine with 1% CHAPS and a pH of 4, both insulin and glucagon demonstrated an adjusted pH (e.g. the insulin pH was 4.61 and adjusted to 3.91, and the glucagon pH was 4.21 and adjusted to 3.84). Both glutamic acid and glycine demonstrated improved solubility for glucagon (>0.5 mg/mL). Compared to glycine & glutamic acid, glucagon demonstrated poor solubility in histidine with CHAPS at pH 4.

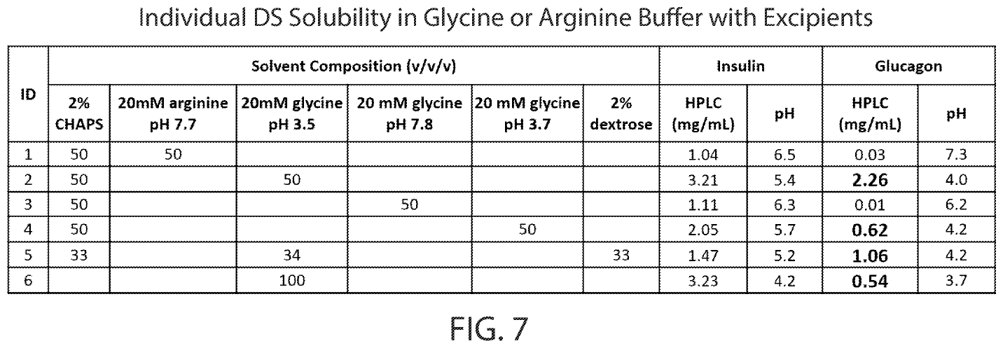

FIG. 7 demonstrates data related to the solubility of insulin (e.g. up to approximately 3.5 mg/mL) and glucagon (e.g. up to approximately 2.5 mg/mL), respectively, in various solutions comprising arginine or glycine, and the addition of an excipient. The arginine solution comprises a pH of 7.7 and the glycine solutions comprise a pH of between 3.5 and 7.8, and further comprising at least one of the following excipients: CHAPS; and dextrose. The concentrations of insulin and glucagon in the supernatant were analyzed by RP-HPLC. Insulin demonstrated a poor solubility of 3.23 mg/mL or less in each of the arginine and glycine solutions. Glucagon demonstrated an improved solubility of 0.54 mg/mL or greater in solutions comprising: 2% CHAPS and 20 mM glycine with a pH of 3.5; 2% CHAPS and 20 mM glycine with a pH of 3.7; 2% CHAPS, 20 mM glycine with a pH of 3.5, and 2% dextrose; and 20 mM glycine with a pH of 7.8.

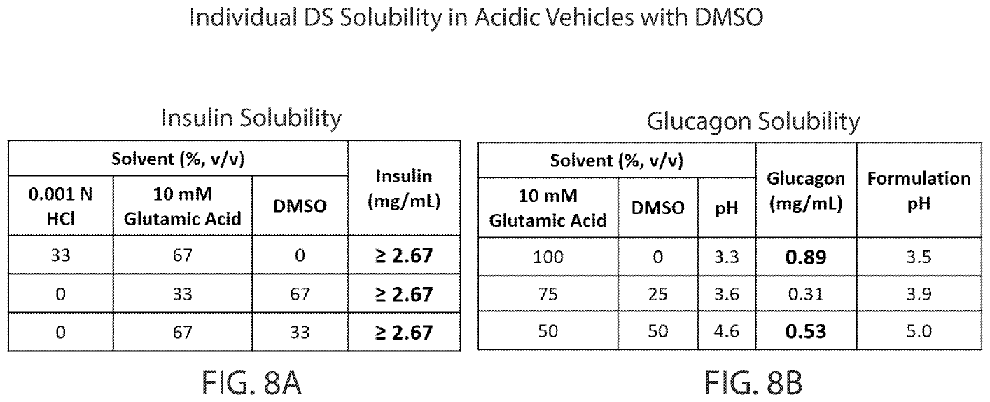

Referring now to FIGS. 8-11, results from studies conducted by applicant are presented, consistent with the present inventive concepts. These studies were conducted to support the development of a co-formulation (e.g. composition 200) of insulin and glucagon containing non-aqueous co-solvent for use in infusion pumps, such as pump 100 of system 10 described hereabove, that is stable at 2-8° C. Challenges included the solubility of insulin and glucagon in non-aqueous solution co-formulation with organic solvents, as well as the co-formulation's compatibility with infusion pump 100. With an objective of achieving improved solubility and stability, two approaches were employed: pure organic solvents, such as acetone, benzyl alcohol, benzyl benzoate, dimethyl sulfoxide (DMSO), ethanol, ethyl acetate, glycerol, methanol, NMP, polyethylene glycol, and propylene glycol (PG); and co-solvents, such as a solution of organic solvents mixed with aqueous solution.

These studies were conducted with a target concentration of insulin of between 1 to 10 mg/mL and a target concentration of glucagon of between 0.1 and 1.0 mg/mL, more specifically an insulin concentration from 3 to 5 mg/mL and a glucagon concentration from 0.1 to 0.8 mg/mL. A basal insulin rate of U-100 between 10-20 μL/hr, and an I/G molar ratio of 3:1 to 16:1 with a fixed insulin dose were assumed.

FIGS. 8A and B illustrate data related to the solubility of insulin and glucagon, respectively, in acidic aqueous solvents with DMSO as a co-solvent. As shown in FIG. 8A, insulin demonstrated an improved solubility of greater than or equal to 2.67 mg/mL in each of the acidic solvents comprising 0-33% hydrochloric acid (HC1), 33-67% glutamic acid, and 0-67% DMSO. As shown in FIG. 8B, glucagon demonstrated an improved solubility of 0.89 mg/mL in a first acidic solvent comprising 100% glutamic acid and 0% DMSO, and an improved solubility of 0.53 mg/mL in a second acidic solvent comprising 50% glutamic acid and 50% DMSO. Glucagon demonstrated a poor solubility of 0.3 lmg/mL in a third acidic solvent comprising 75% glutamic acid and 25% DMSO. Glucagon demonstrated a better solubility in glutamic acid (e.g. a concentration of 0.89 mg/mL) than the inorganic aqueous buffer systems. The addition of DMSO did not increase glucagon solubility in glutamic acid.

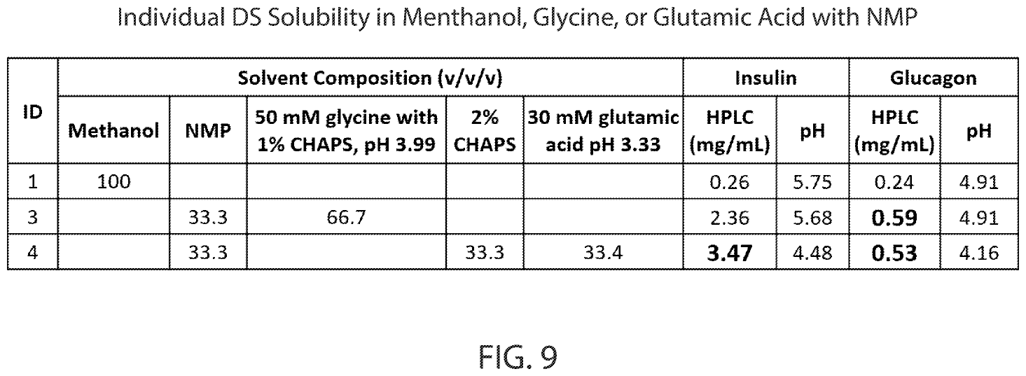

FIG. 9 illustrates data related to the solubility of insulin (e.g. up to approximately 3.3 mg/mL) and glucagon (e.g. up to approximately 1.7 mg/mL), respectively, in solvents comprising at least one of the following: methanol; glycine with a pH of 4; glutamic acid with a pH 3.3; NMP; and CHAPS, consistent with the present inventive concepts. The concentrations of insulin and glucagon in the supernatant were measured by RP-HPLC. Insulin and glucagon demonstrated a poor solubility of less than 0.3 mg/mL in 100% methanol. Additionally, insulin demonstrated a poor solubility of 2.36 mg/mL in solution comprising 33.3% NMP and 66.7% of 50 mM glycine with 1% CHAPS. Insulin demonstrated an improved solubility of 3.47 mg/mL in solution comprising 33.3% NMP, 33.3% of 2% CHAPS, and 33.4% of 30 mM glutamic acid. Glucagon demonstrated an improved solubility of 0.59 mg/mL in solution comprising 33.3% NMP and 66.7% of 50 mM glycine with 1% CHAPS. Additionally, glucagon demonstrated an improved solubility of 0.53 mg/mL in solution comprising 33.3% NMP, 33.3% of 2% CHAPS, and 33.4% of 30 mM glutamic acid.

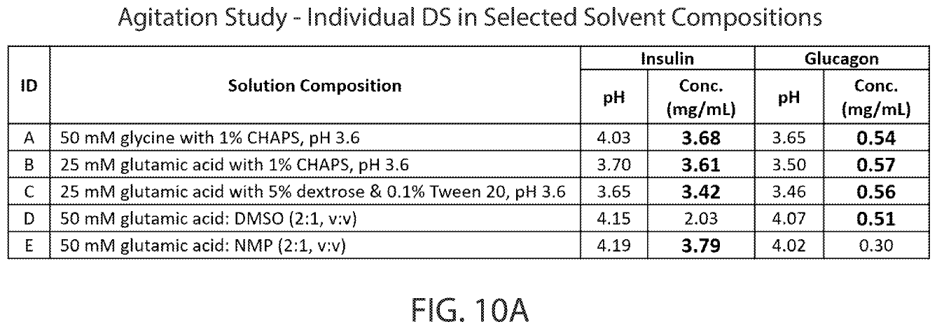

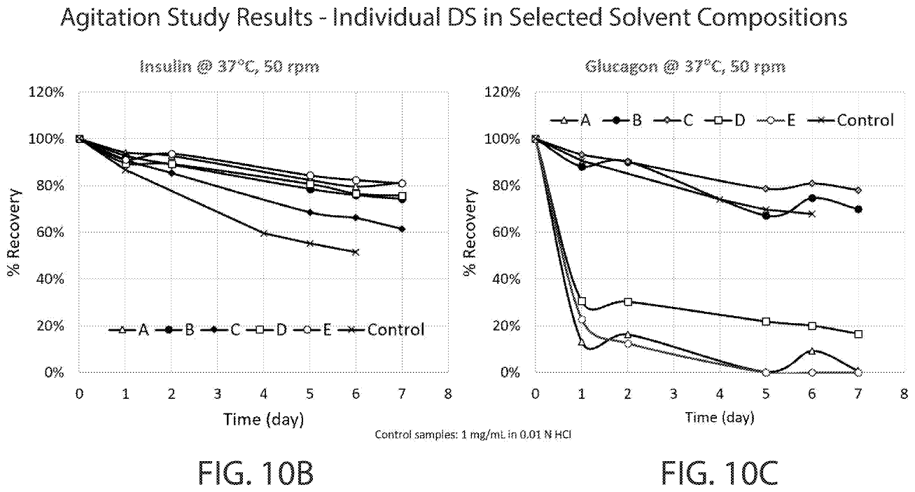

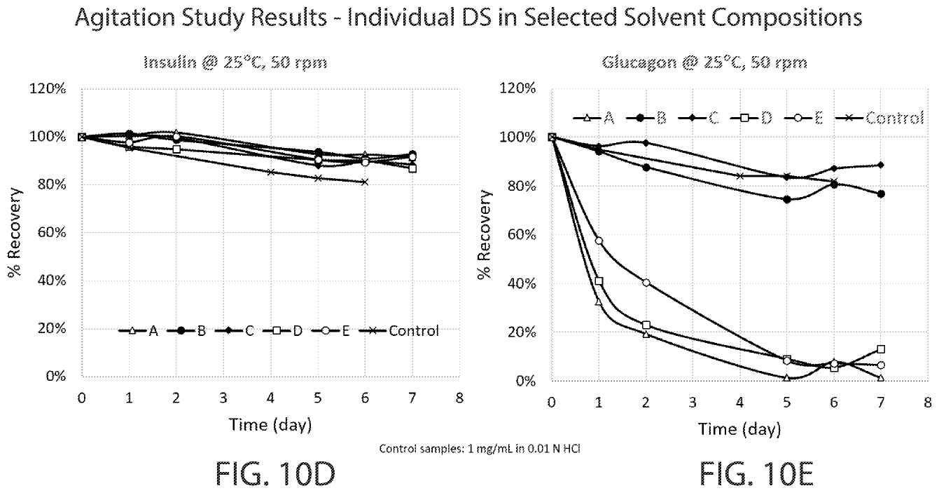

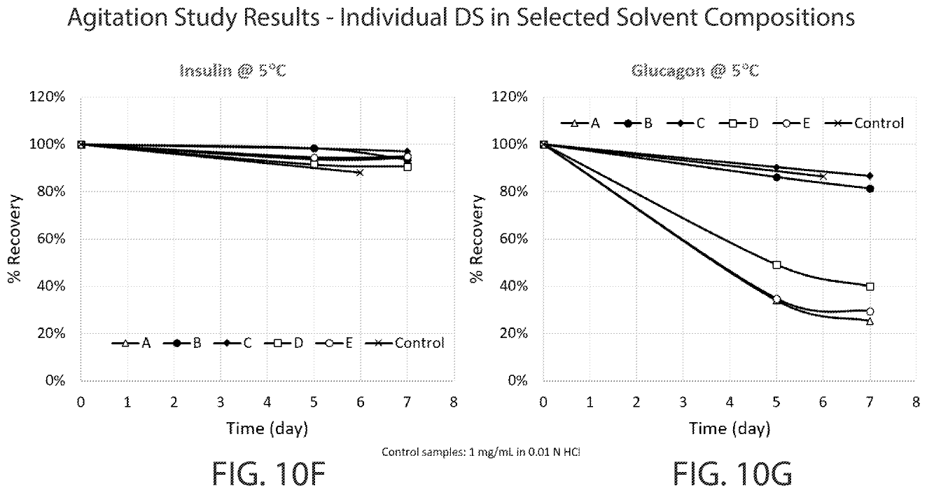

FIGS. 10A-G illustrate data related to an agitation study that was conducted for insulin and glucagon in various solvents: glycine with CHAPS at a pH of 3.6; glutamic acid with CHAPS at a pH of 3.6; glutamic acid with dextrose and Tween 20 at a pH of 3.6; glutamic acid with DMSO; and glutamic acid with NMP. The concentration of insulin ranges from 2.03 to 3.79 mg/mL and the concentration of glucagon ranges from 0.30 to 0.57 mg/mL. Control samples were prepared in 0.01N HCl at 1.0 mg/mL. Aliquoted samples were placed in three conditions up to 7 days: 5° C. with 0 rpm (as control), 25° C. with 50 rpm, and 37° C. with 50 rpm. At each time point, samples were diluted with 0.01N HCl, then analyzed by RP-HPLC. Insulin demonstrated good recovery after 7 days agitation in all solvents tested. Glucagon demonstrated good recovery after 7 days agitation in solvent comprising glutamic acid with CHAPS or with dextrose and Tween 20. In solvent comprising glutamic acid with DMSO or NMP, glucagon demonstrated poor recovery after 7 days agitation.

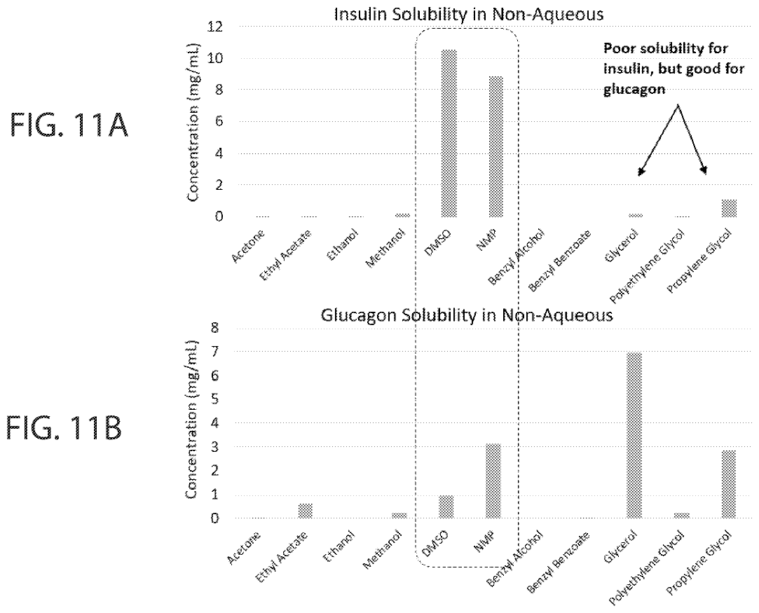

FIGS. 11A and B illustrate data related to the solubility of insulin and glucagon, respectively, in non-aqueous solvents, consistent with the present inventive concepts. The concentrations of insulin and glucagon in the supernatant were analyzed by RP-HPLC. Both DMSO and NMP demonstrated improved solubility for insulin (e.g. a concentration greater than 8 mg/mL) and glucagon (e.g. a concentration greater than 1 mg/mL). Both glycerol and propylene glycol demonstrated improved solubility for glucagon (e.g. a concentration greater than 2.5 mg/mL), but poor solubility for insulin (e.g. a concentration less than 2 mg/mL). Non-aqueous solvents with a dielectric constant between 25 and 70 have a relatively higher solubility for glucagon, for example, solvents such as N-methyl pyrrolidone (32), methanol (33), N,N-dimethyl formamide (37), acetonitrile (38), dimethyl acetamide (38), DMSO (47), propylene carbonate (65).

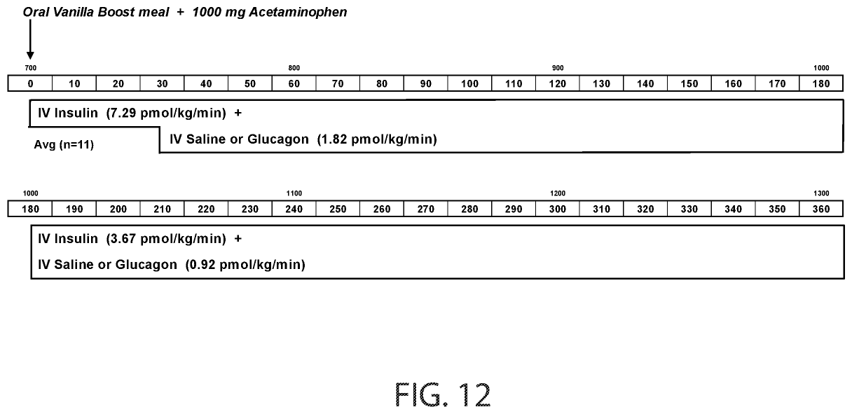

Referring now to FIG. 12, a study protocol conducted by the applicant in human patients with Type 1 diabetes is illustrated, consistent with the present inventive concepts. Applicant conducted a human study protocol for co-administering insulin and glucagon, at a fixed ratio, to patients with Type 1 diabetes. The patients were subjected to a liquid meal challenge during the simultaneous co-administration of the two hormones (insulin and glucagon) which was compared to the same challenge with the patients' receiving insulin administration alone. The liquid meal challenge allows a clinically relevant assessment of plasma glucose rise and fall. During the two study visits, after an overnight fast in which intravenous (IV) insulin was given to normalize plasma glucose level, the mixed meal was administered at time t=0. IV insulin was started at the same time at an average rate of 7.29 pmol/kg/min, although the rate for each individual patient was determined by their overnight insulin infusion dose and their individual insulin treatment history. At time t=30 minutes, an infusion of either saline or glucagon (1.82 pmol/kg/min) was started during the two treatment studies. Glucagon was purposefully started 30 minutes after the time of insulin delivery (where insulin was given at time t=0) as earlier non-clinical work had established that glucagon had a more rapid onset of action than insulin in its respective actions to regulate plasma glucose, and the 30 minute delay was applied to better synchronize the pharmacological effects of insulin and glucagon. At time t=180 minutes, the insulin and glucagon infusion rates were both halved. Therefore, the individual glucagon infusion rates varied with the insulin infusion rates in order to maintain an insulin-glucagon ratio (Ins/GGN ratio) of 4:1 throughout each experiment (with the exception of the first 30 minutes of the study). By protocol, if plasma glucose dropped to 50 mg/dl, a variable glucose infusion was applied to maintain the glucose level at approximately 50 mg/dl, to avoid more serious hypoglycemia from occurring.

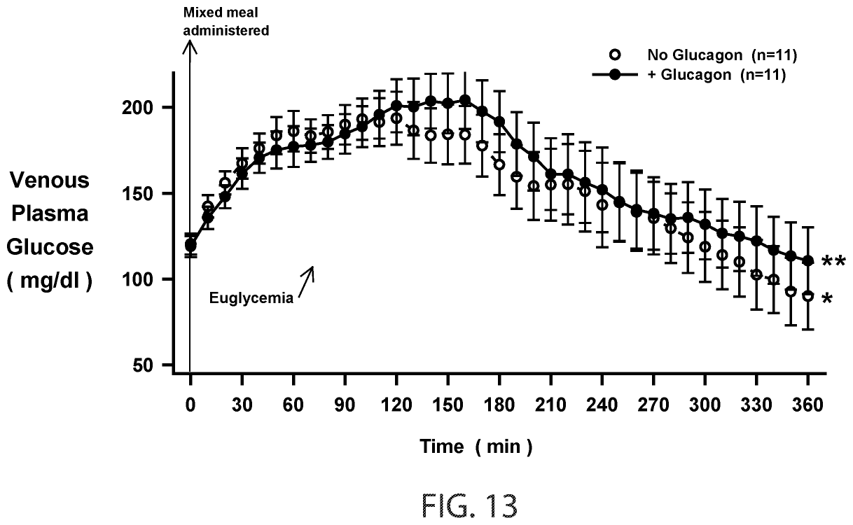

Referring now to FIGS. 13-20, data collected from studies conducted by the applicant in human patients with Type 1 diabetes are illustrated, consistent with the present inventive concepts.

FIG. 13 illustrates the glucose excursion following the meal challenge for eleven patients (also referred to as “subjects” herein). Per protocol, in randomized order, each patient was admininstered either IV insulin infusion (shown as “No Glucagon”), or IV insulin and IV glucagon co-infusions (shown as “+Glucagon”) at the pre-detemined fixed ratio. The glucose excursion data is also presented below in Table 1, which compares peak plasma glucose levels and AUCs (area under curve) for the two treatment groups.

TABLE 1

| PLASMA GLUCOSE RISE | Insulin-Alone | Insulin-Glucagon |
|---|---|---|
| Fasting PG (mg/dl | 121 ± 5 | 119 ± 6 |
| Peak PG (mg/dl) | 224 ± 18 | 228 ± 15 |
| ΔPG 0-120_{AUC} (mg · min/dl) | 8162 ± 1514 | 7871 ± 926 |
| ΔPG 0-180_{AUC} (mg · min/dl) | 10501 ± 3014 | 11536 ± 1778 |

| PLASMA GLUCOSE DECLINE | Insulin-Alone | Insulin-Glucagon |
|---|---|---|
| PG nadir (mg/dl)* | 51 ± 4.0 | 114 ± 12.0** |
| Requiring IV glucose rescue | 5/11 | 2/11 |
| Time <70 mg/dl (min) | 20 ± 7% | 9 ± 4%* |

**contrasting PG levels at lowest point during insulin-alone arm at equivalent time point during insulin-glucagon arm.
**p = 0.007
*statistical significance p = 0.013 between treatment arms

The glucose profiles that resulted indicate that the insulin-glucagon combination allows a robust protection against problematic hypoglycemia without significant worsening of hyperglycemia. Five of eleven insulin-alone (“No Glucagon”) treated subjects had to be rescued with glucose infusion (the average glucose infusion rate over the last 30 minutes in all subjects was 3.8 +/−1.6 mg/kg/min). In contrast, only two of the eleven insulin-glucagon (“+Glucagon”) treated subjects had to be rescued with glucose infusion (the average glucose infusion rate over the last 30 minutes in those two subjects averaged 1.1 +/−0.8 mg/kg/min).

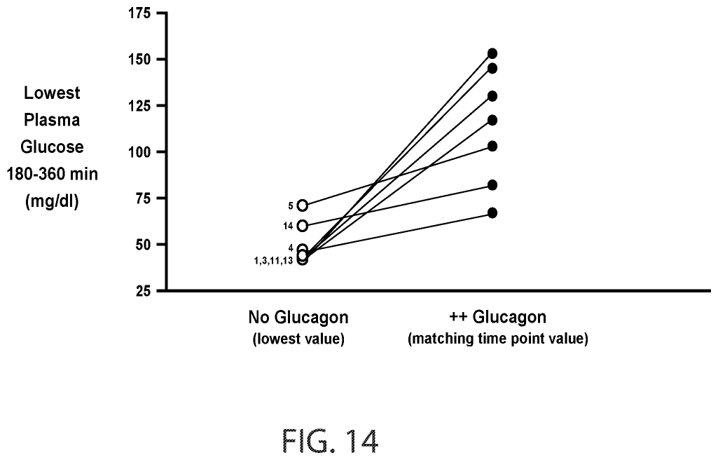

FIG. 14 illustrates the hypoglycemia-protective effect of the co-administered insulin-glucagon approach by comparing the plasma glucose nadir observed in the seven subjects who experienced an abnormal lowering of plasma glucose (<80 mg/dl) in the insulin-alone treatment arm with the plasma glucose level observed at the matching time point in the insulin-glucagon treatment arm. In all seven subjects, insulin-glucagon treatment conferred hypoglycemia protection with an average plasma glucose nadir in the insulin-alone treatment arm of 50±4 mg/dl and 114±12 mgdl in the insulin-glucagon treatment arm. Additionally, a comparison of the average plasma glucose level during the last 30 minutes of the experiment (5 1/2 hours after administration of liquid meal) in the 7 subjects whose glucose fell below the pre-meal value in the last 30 minutes of the experiment was made. In the insulin-alone group (“No Glucagon”), the 5 subjects with the lowest glucose levels had to be rescued (average glucose infusion rate of 5.9 +/−2.2 mg/kg/min). In the insulin-glucagon group (“+Glucagon), only 2 of 7 subjects had to be rescued (average glucose infusion rate of 1.8 +/−1.1 mg/kg/min).

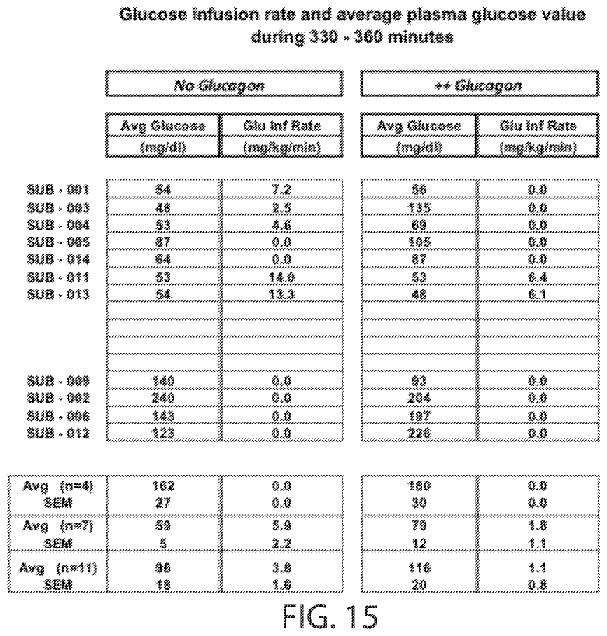

FIG. 15 illustrates the rescue glucose infusion rates and average plasma glucose values for the eleven patients during the 330-360 minute interval of the study protocol. The data provides further evidence of the hypoglycemia-protective effect of the insulin-glucagon combination viewed from an individual patient basis.

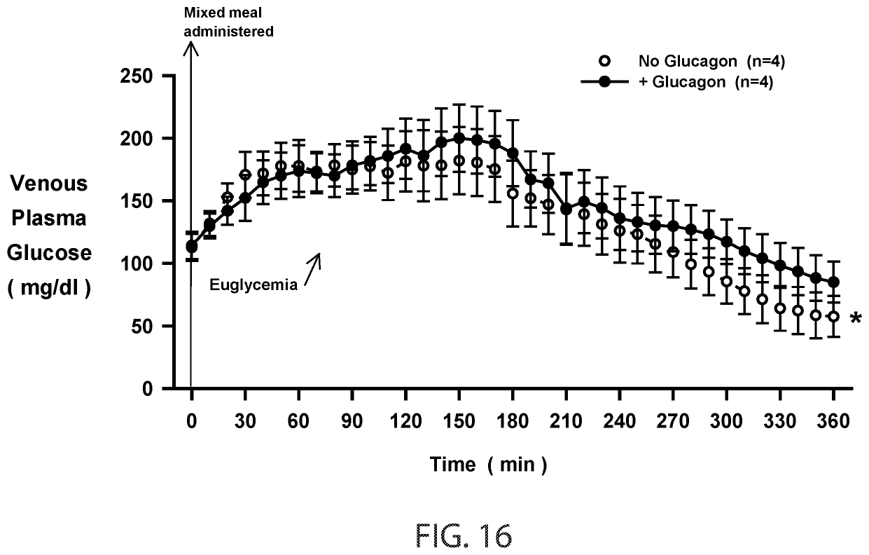

Referring specifically to FIGS. 16-20, plasma glucose curves, pharmacokinetic profiles of insulin and glucagon and sympathetic nervous system responses are described from a subset of four patients.

FIG. 16 illustrates the rise and fall of plasma glucose for the four patients who had undergone a more complete hormone analysis when treated with either insulin-alone or with the insulin-glucagon combination. Three out of the four patients in the insulin-alone treatment arm had to be rescued with glucose infusions, whereas no glucose infusion rescue was required for the patients in the insulin-glucagon treatment arm.

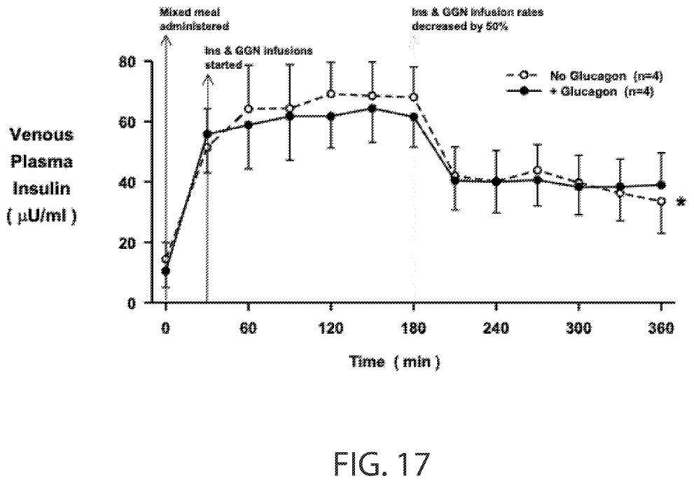

FIG. 17 illustrates that the venous plasma insulin levels were very similar for the insulin-alone treatment and the insulin-glucagon combination treatment over the 360 min time period for the four patients. Three out of the four patients in the insulin-alone treatment arm had to be rescued with glucose infusions.

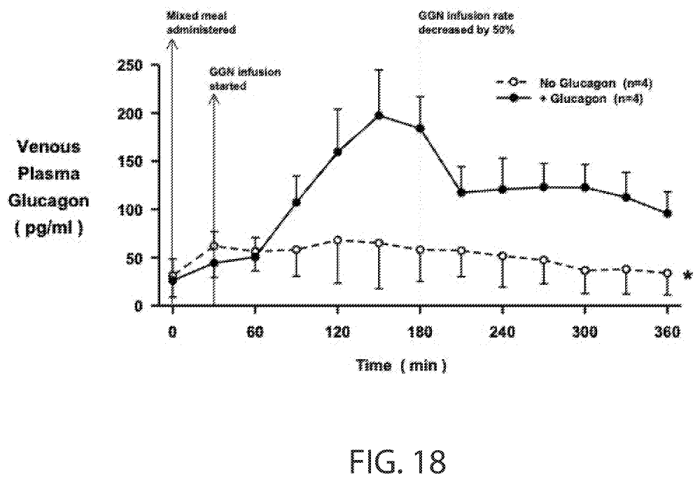

FIG. 18 illustrates that the plasma glucagon levels during the insulin-glucagon combination treatment arm resulted in the expected elevation of glucagon levels (approximately 4-5 fold higher than basal or fasting glucagon levels) compared to the insulin-alone treatment arm per protocol over the 30-360 time period. The glucagon infusion rate was 8-10×basal during the 0-180 minute time period and 4-5×basal during the 180-360 minute time period. Three out of the four patients in the insulin-alone treatment arm had to be rescued with glucose infusions.

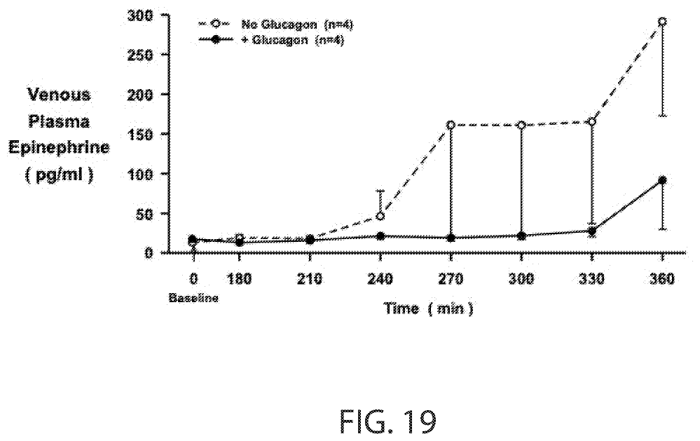

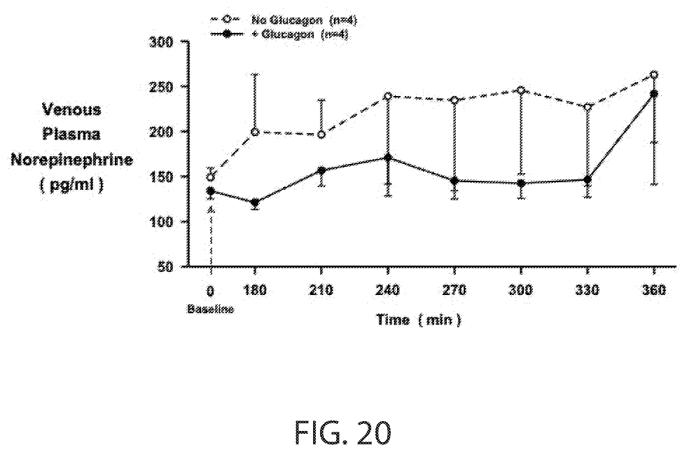

Figs.19 and 20 illustrate that the four patients on insulin-alone treatment display a sympathetic central nervous system response to hypoglycemia through elevations of venous plasma norepinephrine and epinephrine, whereas the insulin-glucagon treatment blunts this autonomic response. Three out of the four patients in the insulin-alone treatment arm had to be rescued with glucose infusions. These studies inidcate that in the presence of elevated plasma glucagon, hypoglycemic exposure is greatly reduced, and as such, reduces the sympathetic nervous system response which may be due to an attenuated hypoglycemic stressor (counter-regulatory) response or through a glucose-independent effect of glucagon itself, or some combination of both. It is conceivable this sparing of the sympathetic nervous system could allow for more counter-regulatory (stressor) reserve in the instance of a further decline in plasma glucose, thereby providing greater protection against hypoglycemia. The studies demonstrate that a co-administered or a co-formulated combined insulin and glucagon solution of the present inventive concepts can provide safe and effective therapeutic value to a diabetic patient

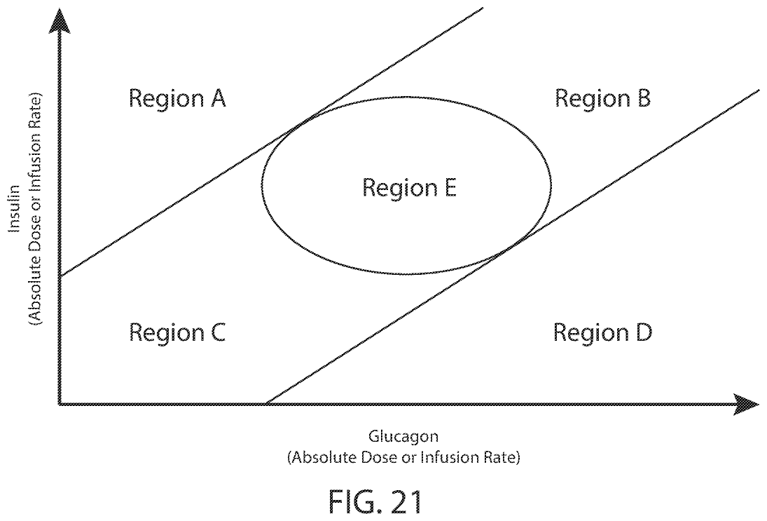

Referring now to FIG. 21, a graph demonstrating optimal insulin to glucagon molar ratios and dosages is illustrated. A certain amount of insulin is required in the treatment of diabetes, otherwise hyperglycemia cannot be effectively controlled. Insulin dominates glucagon at higher doses (e.g. absolute doses) due to the different opposing actions of the two peptides. Insulin at higher doses regulates blood glucose, whereas glucagon does not. Glucagon at higher doses effects a toxicity, whereas insulin does not. As shown, five regions, Regions A-E, indicate various treatment outcomes related to the dosage of both insulin and glucagon. Outcomes are based on both the ratio of insulin to glucagon, as well as the absolute amount of the dose (e.g. an average amount delivered over a time period, such as a time period of at least 1 minute, 5 minutes, 15 minutes, 30 minutes, and/or 60 minutes) of each peptide. Region A illustrates ratios and dosages representing a high insulin dose and a low glucagon dose, whereby maintaining these dosages results in insulin dominating the effects of glucagon, and an ability to control hyperglycemia but potential inability to control hypoglycemia. Region B illustrates ratios and dosages representing a high insulin dose and a high glucaon dose, whereby maintaining these dosages results in insulin dominating the effects of glucagon, and the potential for hypoglcycemia and/or glucagon toxicitiy being enhanced. Region C illustrates ratios and dosages representing a low insulin dose and a low glucagon dose (e.g. a minimal amount of insulin independent of the amount of glucagon), whereby maintaining these dosages results in glucagon dominating the effects of insulin, and a potential inablility to control hyperglycemia. Region D illustrates ratios and dosages representing a low insulin dose and a high glucagon dose, whereby maintaining these dosages results in glucagon dominating the effects of insulin, with the potential for hyperglcycemia and/or glucagon toxictiy being enhanced. Region E illustrates an optimal set of ratios and dosages for the insulin dose and glucagon dose (e.g. optimal dose and ratio) proposed by the Applicant, comprising a moderate insulin dose and a moderate glucagon dose, whereby maintaining these dosages results in a balance of insulin to glucagon, and the ability to control (e.g. avoid) both hyperglycemia and hypoglycemia.

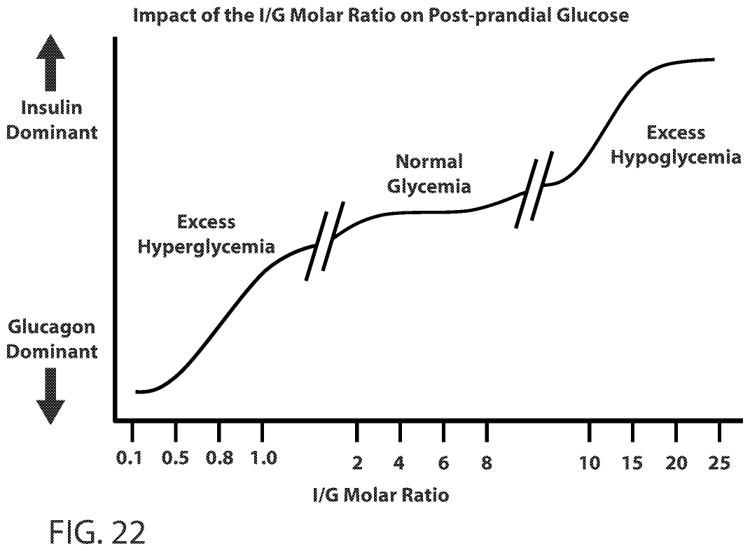

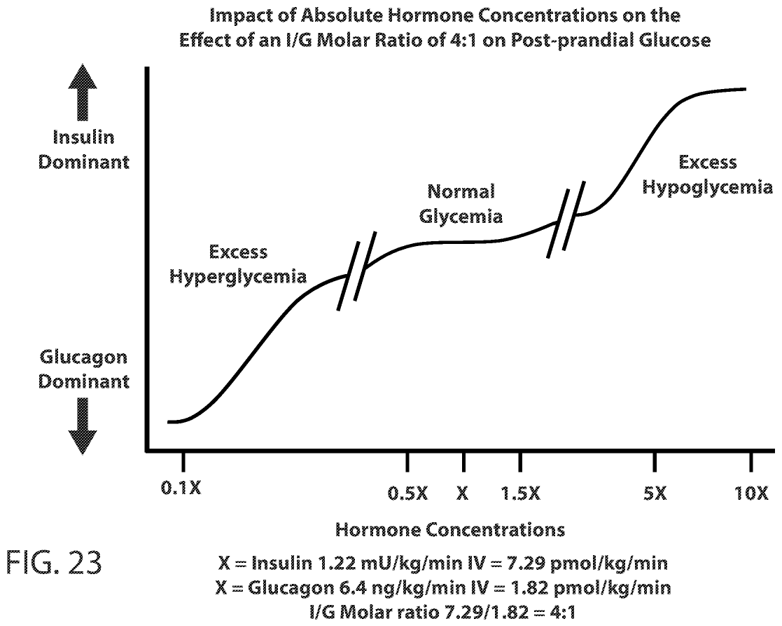

Referring now to FIG. 22, a graph demonstrating the impact of various insulin to glucagon molar ratios on post-prandial glucose is illustrated. The graph shows the ability of a given insulin-glucagon molar ratio to maintain normal post-prandial glycemia while at the same time protecting against post-meal hypoglycemia (i.e. an I/G molar ratio in the range of 1:1 to 8:1). If the I/G molar ratio exceeds 8:1, the amount of insulin can overcome glucagon's protective effect against low blood sugar (hypoglycemia). If the I/G molar ratio is less than 1:1, glucagon can worsen post-prandial hyperglycemia. As shown in FIG. 23 herebelow, an optimized I/G molar ratio can only be efficacious (e.g. safe and efficacious) if the appropriate rates of insulin and glucagon infusion are delivered to the patient (e.g. minimum and/or maximum rates delivered at the optimized I/G molar ratio). In some embodiments, insulin and glucagon are delivered (e.g. a co-formulation of insulin and glucagon is delivered) such that the average amount of each hormone delivered over a time period (e.g. at any delivery rate) is maintained above a minimum threshold and/or below a maximum threshold (e.g. the minimum and maximum thresholds described herebelow in reference to FIG. 23).

Referring now to FIG. 23, a graph demonstrating the impact of varous insulin and glucose absolute concentrations on post-prandial glucose is illustrated. The efficacy of a given I/G molar ratio is not independent of the absolute hormone concentrations delivered to the patient (e.g. over a time period). An I/G molar ratio of 4:1 comprised of insulin being delivered at a rate (e.g. an average rate) between 3.65 pmol/kg/min to 10.95 pmol/kg/min and glucagon being delivered at a rate (e.g. an average rate) between 0.91 pmol/kg/min to 2.74 pmol/kg/min can result in both control of post-prandial glycemia, as well as limiting of post-meal hypoglycemia. The optimized minimum and maximum rates of infusion (e.g. average rates of infusion) reflect the patient's metabolic state. When the absolute amounts of the hormones infused into the patient reaches five times the optimal rate (in the illustrated example, five times an optimal IV insulin infusion rate of 7.29 pmol/kg/min and five times an optimal glucagon infusion rate of 1.82 pmol/kg/min) insulin can dominate and post-meal hypoglycemia is likely. When the infusion rates of the delivery of the hormones drop below 0.5 times the optimal rates, glucagon can dominate and excessive post-prandial hyperglycemia is likely. The systems, devices, compositions, and methods of the present inventive concepts can be configured to maintain delivery of these two hormones (e.g. average delivery of a co-formulation of insulin and glucagon over a period of time such as 1 minute, 5 minutes, 15 minutes, 30 minutes, and/or 60 minutes) above the minimum and below the maximums described hereabove. In some embodiments, delivery of the hormones includes delivery for a first time period at a first rate, and delivery for a second time period at a second rate. The first rate can comprise a rate that is at a level near or even above the above maximum rate described hereabove, and the second rate can comprise a delivery rate that is at or near zero (e.g. such that the average rate of delivery over a time period, such as a time period comprising the first time period plus the second time period, results in an average delivery rate between the minimum and maximum rates described hereabove).

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A method of treating diabetes in a patient, the method comprising:
co-administering insulin and glucagon to the patient, and
wherein the insulin and glucagon are co-administrated at an insulin: glucagon molar ratio of between 1:1 and 8:1, such as a ratio between 3:1 and 4:1,
wherein the insulin and glucagon are administered in an amount therapeutically effective to simultaneously treat and/or inhibit at least one of hyperglycemia and hypoglycemia.

2.-61. (canceled)
